(19) United States
(12) Patent Application Publication
(10) Pub. No.: US 2007/0010565 A1
(43) Pub. Date: Jan. 11, 2007

(54) New thiazolidinones without basic nitrogen, their production and use as pharmaceutical agents

(76) Inventors: Olaf Prien, Berlin (DE); Volker Schulze, Bergfelde (DE); Knut Eis, Berlin (DE); Lars Wortmann, Berlin (DE); Dirk Kosemund, Erfurt (DE); Gerhard Siemeister, Berlin (DE); Uwe Eberspaecher, Berlin (DE); Judith Guenther, Berlin (DE); Dominic E.A. Brittain, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/410,284

(22) Filed: Apr. 25, 2006

Related U.S. Application Data
(60) Provisional application No. 60/676,947, filed on May 3, 2005.

(30) Foreign Application Priority Data
Apr. 25, 2005 (DE) 102005020105.9

(51) Int. Cl. A61K 31/426

(52) U.S. Cl. 514/369; 548/185

(57) ABSTRACT

The invention relates to thiazolidones of general formula (I): 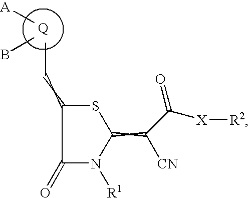 to their production and to their use as inhibitors of the polo-like kinase (Plk) for treating various diseases.

This application claims the benefit of the filing date of U.S. Provisional Application Ser. No. 60/676,947 filed May 3, 2005.

The invention relates to thiazolidinones, to their production and to their use as inhibitors of polo-like kinases (Plk) for treating various diseases.

Tumour cells are distinguished by an uninhibited cell-cycle process. On the one hand, this is based on the loss of control proteins, such as RB, p16, p21, p53, etc., as well as the activation of so-called accelerators of the cell-cycle process, the cyclin-dependent kinases (Cdks). The Cdks are an anti-tumour target protein that is acknowledged in pharmaceutics. In addition to the Cdks, serine/threonine kinases that regulate the new cell cycle, so-called ‘polo-like kinases,’ were described, which are involved not only in the regulation of the cell cycle but also in the coordination with other processes during mitosis and cytokinesis (formation of the spindle apparatus, chromosome separation). This class of proteins therefore represents an advantageous point of application for therapeutic intervention of proliferative diseases such as cancer (Descombes and Nigg. Embo J, 17; 1328 et seq., 1998; Glover et al. Genes Dev 12, 3777 et seq., 1998).

A high expression rate of Plk-1 was found in ‘non-small cell lung’ cancer (Wolf et al. Oncogene, 14, 543 et seq., 1997), in melanomas (Strebhardt et al. JAMA, 283, 479 et seq., 2000), in ‘squamous cell carcinomas’ (Knecht et al. Cancer Res, 59, 2794 et seq., 1999) and in ‘esophageal carcinomas’ (Tokumitsu et at. Int J Oncol 15, 687 et seq., 1999).

A correlation of a high expression rate in tumour patients with poor prognosis was shown for the most varied tumours (Strebhardt et al. JAMA, 283, 479 et seq., 2000, Knecht et at. Cancer Res, 59, 2794 et seq., 1999 and Tokumitsu et al. Int J Oncol 15, 687 et seq., 1999).

The constitutive expression of Plk-1 in NIH-3T3 cells resulted in a malignant transformation (increased proliferation, growth in soft agar, colony formation and tumour development in hairless mice) (Smith et al. Biochem Biophys Res Comm, 234, 397 et seq., 1997).

Microinjections of Plk-1 antibodies in HeLa cells resulted in improper mitosis (Lane et al.; Journal Cell Biol, 135, 1701 et seq., 1996).

With a ‘20-mer’ antisense oligo, it was possible to inhibit the expression of Plk-1 in A549 cells, and to stop their ability to survive. It was also possible to show a significant anti-tumour action in hairless mice (Mundt et at., Biochem Biophys Res Comm, 269, 377 et seq., 2000).

The microinjection of anti-Plk antibodies in non-immortalized human Hs68 cells showed, in comparison to HeLa cells, a significantly higher fraction of cells, which remained in a growth arrest at G2 and showed far fewer signs of improper mitosis (Lane et al.; Journal Cell Biol, 135, 1701 et seq., 1996).

In contrast to tumour cells, antisense-oligo-molecules did not inhibit the growth and the viability of primary human mesangial cells (Mundt et at., Biochem Biophys Res Comm, 269, 377 et seq., 2000).

In mammals, to date in addition to the Plk-1, three other polo-kinases were described that are induced as a mitogenic response and exert their function in the G1 phase of the cell cycle. These are, on the one hand, the so-called Prk/Plk-3 (the human homolog of the mouse-Fnk=fibroblast growth factor-induced kinase; Wiest et at, Genes, Chromosomes & Cancer, 32: 384 et seq., 2001), Snk/Plk-2 (Serum-Induced Kinase, Liby et at., DNA Sequence, 11, 527-33, 2001) and sak/Plk4 (Fode et at., Proc. Natl. Acad. Sci. U.S.A., 91, 6388 et seq; 1994).

The inhibition of Plk-1 and the other kinases of the polo family, such as Plk-2, Plk-3 and Plk-4, thus represents a promising approach for the treatment of various diseases.

The sequence identity within the Plk domains of the polo family is between 40 and 60%, so that partial interaction of inhibitors of a kinase occurs with one or more other kinases of this family. Depending on the structure of the inhibitor, however, the action can also take place selectively or preferably on only one kinase of the polo family.

In International Application WO 03/093249, thiazolidinone compounds that inhibit the kinases of the polo family are disclosed.

The object of this invention is now to make available additional substances that inhibit kinases of the polo family in the micro- and nanomolar range.

It has now been found that compounds of general formula I: 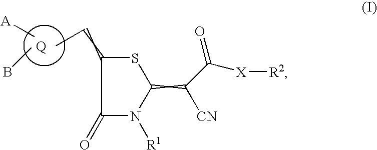
in which
Q stands for aryl,
A and B, independently of one another, stand for hydrogen, halogen, hydroxy, NR³R⁴, cyano or nitro,
   or
   for C₁-C₄-alkyl, C₁-C₆-alkoxy or C₃-C₆-heterocycloalkyl that optionally is substituted in one or more places, in the same way or differently, with halogen, hydroxy, C₃-C₆-heterocycloalkyl or with the group —NR³R⁴ or —CO(NR³)-M, whereby the heterocycloalkyl itself optionally can be interrupted by one or more nitrogen, oxygen and/or sulfur atoms and/or optionally can be interrupted by one or more —C(O)— or —SO₂— groups in the ring and/or optionally one or more double bonds can be contained in the ring and/or the ring itself optionally can be substituted in one or more places, in the same way or differently, with C₁-C₆-alkyl, C₃-C₆-cycloalkyl, C₁-C₆-hydroxyalkyl or with the group —NR³R⁴,
   or
   for —NR³C(O)-L, —NR³C(O)—NR³-L, —COR⁶, —O—(CH₂)ₚR⁶, —CO(NR³)-M, —NR³(CS)NR³R⁴, —NR³SO₂-M, —SO₂—NR³R⁴, —SO₂(NR³)-M or —O—(CH₂)ₚaryl,
p stands for an integer of 0, 1, 2, 3, or 4,
L stands for C₁-C₆-alkyl or C₃-C₆-heterocycloalkyl that optionally is substituted in one or more places, in the same way or differently, with C₁-C₆-hydroxyalkoxy, C₁-C₆-alkoxyalkoxy, C₃-C₆-heterocycloalkyl or with the group —NR³R⁴, whereby the heterocycloalkyl itself optionally can be interrupted by one or more nitrogen, oxygen and/or sulfur atoms and/or optionally can be interrupted by one or more —C(O)— or —SO₂— groups in the ring and/or optionally one or more double bonds can be contained in the ring, and/or the ring itself optionally can be substituted in one or more places, in the same way or differently, with C₁-C₆-alkyl, C₃-C₆-cycloalkyl, C₁-C₆-hydroxyalkyl or with the group —NR³R⁴,
M stands for C₁-C₆-alkyl that optionally is substituted in one or more places, in the same way or differently, with the group —NR³R⁴ or C₃-C₆-heterocycloalkyl,
X stands for —NH— or —NR⁵—,
R¹ stands for C₁-C₄-alkyl, C₃-cycloalkyl, allyl or propargyl that optionally is substituted in one or more places, in the same way or differently, with halogen,
R² stands for hydrogen or for C₁-C₆-alkyl, C₁-C₆-alkoxy, C₁-C₆-alkenyl, C₁-C₆-alkynyl, C₃-C₆-cycloalkyl, C₃-C₆-heterocycloalkyl, aryl or heteroaryl that optionally is substituted in one or more places, in the same way or differently, with halogen, hydroxy, cyano, C₁-C₆-alkyl, C₁-C₆-alkoxy, C₁-C₆-hydroxyalkyl, C₃-C₆-cycloalkyl, C₃-C₆-heterocycloalkyl, C₁-C₆-alkynyl, aryl, aryloxy, heteroaryl or with the group —S—C₁-C₆-alkyl, —COR⁶, —NR³R⁴, —NR³C(O)-L or —NR³COOR⁷,
   whereby the heterocycloalkyl itself optionally can be interrupted by one or more nitrogen, oxygen and/or sulfur atoms and/or optionally can be interrupted by one or more —C(O)— or —SO₂ groups in the ring and/or optionally one or more double bonds can be contained in the ring,
   and whereby aryl, heteroaryl, C₃-C₆-cycloalkyl- and/or the C₃-C₆-heterocycloalkyl ring in each case itself optionally can be substituted in one or more places, in the same way or differently, with cyano, halogen, hydroxy, C₁l-C₆-alkyl, C₁-C₆-hydroxyalkyl, or C₁-C₆-alkoxy, C₃-C₆-cycloalkyl, C₃-C₆-heterocycloalkyl, aryl, benzyl or heteroaryl that optionally is substituted in one or more places, in the same way or differently, with halogen,
   or
   for the group —NR³R⁴, —NR³C(O)-L, or —NR³(CS)NR³R⁴,
   or
R² and R⁵ together form a C₃-C₆-heterocycloalkyl ring, which is interrupted at least once by nitrogen and optionally can be interrupted in one or more places by oxygen or sulfur and/or optionally can be interrupted by one or more —C(O)— or —SO₂— groups in the ring and/or optionally one or more double bonds can be contained in the ring,
   and/or the ring itself optionally can be substituted in one or more places, in the same way or differently, with cyano, halogen, hydroxy, C₁-C₆-alkyl, C₃-C₆-cycloalkyl, C₁-C₆-hydroxyalkyl, C₁-C₆-alkoxyalkyl or with the group —NR³R⁴ or —COR⁶,
   and/or can be substituted with aryl or heteroaryl that optionally is substituted in one or more places, in the same way or differently, with halogen, C₁-C₆-alkoxy or with the group —COR⁶,
R³ and R⁴, independently of one another, stand for hydrogen or for C₁-C₆-alkyl, C₁-C₆-alkoxy, —CO—C₁-C₆-alkyl or aryl that optionally is substituted in one or more places, in the same way or differently, with halogen, hydroxy, C₃-C₆-heterocycloalkyl, C₁-C₆-hydroxyalkoxy or with the group —NR³R⁴, whereby the heterocycloalkyl itself optionally can be interrupted by one or more nitrogen, oxygen and/or sulfur atoms and/or optionally can be interrupted by one or more —C(O)— or —SO₂— groups in the ring and/or optionally one or more double bonds can be contained in the ring, and whereby the C₃-C₆-heterocycloalkyl ring itself optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group —NR³R⁴ or —CO—NR³R⁴,
   or
R³ and R⁴ together form a C₃-C₆-heterocycloalkyl ring, which is interrupted at least once by nitrogen and optionally can be interrupted in one or more places by oxygen or sulfur and/or optionally can be interrupted by one or more —C(O)— or —SO₂— groups in the ring and/or optionally one or more double bonds can be contained in the ring,
   and/or the heterocycloalkyl ring itself optionally can be substituted in one or more places, in the same way or differently, with C₁-C₆-alkyl, C₃-C₆-cycloalkyl, C₁-C₆-hydroxyalkyl, C₁-C₆-alkoxyalkyl, cyano, hydroxy or with the group —NR³R⁴,
R⁵ stands for C₁-C₆-alkyl, C₁-C₆-alkenyl, or C₁-C₆-alkynyl that optionally is substituted in one or more places, in the same way or differently, with halogen, hydroxy, cyano, C₁-C₆-alkoxy, C₃-C₆-cycloalkyl, C₃-C₆-heterocycloalkyl, or with the group —NR³R⁴, whereby the heterocycloalkyl itself optionally can be interrupted by one or more nitrogen, oxygen and/or sulfur atoms and/or optionally can be interrupted by one or more —C(O)— or —SO₂ groups in the ring and/or optionally one or more double bonds can be contained in the ring, and
   whereby the C₃-C₆-heterocycloalkyl ring itself in each case optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group —NR³R⁴ or —CO—NR³R⁴,
R⁶ stands for hydroxy, C₁-C₆-alkyl, C₁-C₆-alkoxy or the group —NR³R⁴,
R⁷ stands for —(CH₂)ₙ-aryl or —(CH₂)ₙ-heteroaryl, and
n stands for an integer of 1, 2, 3, 4, 5 or 6,
   as well as the solvates, hydrates, stereoisomers, diastereomers, enantiomers and salts thereof,
   are suitable inhibitors of the kinases of the polo family.

This is surprising, since compounds of general formula I do not have the donor-acceptor motif of the generally known kinase inhibitors that is quite well known and well established from the literature (cf. Structure 1999, Vol. 3, pp. 319, and Science 1998, Vol. 281, p. 533) and that makes possible adequate binding to the hinge region in the catalytic center of the kinase. It is therefore possible, but not absolutely necessary, that compounds of general formula I bind in some other way to the kinases and cause such an inhibitory action.

The compounds of general formula I according to the invention essentially inhibit the polo-like kinases, upon which is based their action against, for example, cancer, such as solid tumours and leukemia; auto-immune diseases, such as psoriasis, alopecia, and multiple sclerosis, chemotherapy agent-induced alopecia and mucositis; cardiovascular diseases, such as stenoses, arterioscleroses and restenoses; infectious diseases, such as those, e.g., produced by unicellular parasites, such as trypanosoma, toxoplasma or plasmodium, or produced by fungi; nephrological diseases, such as, e.g., glomerulonephritis; chronic neurodegenerative diseases, such as Huntington's disease, amyotropic lateral sclerosis, Parkinson's disease, AIDS dementia and Alzheimer's disease; acute neurodegenerative diseases, such as ischemias of the brain and neurotraumas; viral infections, such as, e.g., cytomegalic infections, herpes, hepatitis B and C, and HIV diseases.

Stereoisomers are defined as E/Z- and R/S-isomers as well as mixtures that consist of E/Z- and R/S-isomers.

The following terms used in the decription and the claims have preferably the following meanings

The term “alkyl” is defined in each case as a straight-chain or branched alkyl radical, such as, for example, methyl, ethyl, propyl, isopropyl, butyl, isobutyl, sec.-butyl, tert.-butyl, pentyl, isopentyl, hexyl, heptyl, octyl, nonyl and decyl, and the isomers thereof.

The term “alkoxy” is defined in each case as a straight-chain or branched alkoxy radical, such as, for example, methoxy, ethoxy, propyloxy, isopropyloxy, butyloxy, isobutyloxy, sec.-butyloxy, tert-butyloxy, pentyloxy, isopentyloxy, hexyloxy, heptyloxy, octyloxy, nonyloxy or decyloxy, and the isomers thereof.

The term “alkenyl” is defined in each case as a straight-chain or branched alkenyl group, whereby, for example, the following radicals are meant: vinyl, propen-1-yl, propen-2-yl, but-1-en-1-yl, but-1-en-2-yl, but-2-en-1-yl, but-2-en-2-yl, 2-methyl-prop-2-en-1-yl, 2-methyl-prop-1-en-1-yl, but-1-en-3-yl, but-3-en-1-yl, and allyl.

The term “alkynyl” is defined in each case as a straight-chain or branched alkynyl radical that contains 2 to 6, preferably 2 to 4, C atoms. For example, the following radicals can be mentioned: acetylene, propyn-1-yl, propyn-3-yl, but-1-yn-1-yl, but-1-yn-4-yl, but-2-yn-1-yl, but-1-yn-3-yl, etc.

The term “heterocycloalkyl” stands for an alkyl ring that comprises 3 to 6 carbon atoms, in which one or more carbon contains is (are) replaced by one or more heteroatoms that are the same or different, such as, e.g., oxygen, sulfur or nitrogen and/or optionally can be interrupted by one or more —C(O)— or —SO₂— groups in the ring, and/or optionally one or more double bonds can be contained in the ring, and can contain another substituent on one or more carbon, nitrogen or sulfur atoms, optionally independently of one another. Substituents on the heterocycloalkyl ring can be: cyano, halogen, hydroxy, C₁-C₆-alkyl, C₁-C₆-alkoxy, C₁-C₆-alkoxyalkyl, C₁-C₆-hydroxyalkyl, C₃-C₆-cycloalkyl, aryl, or the group —NR³R⁴, —CO—NR³R⁴, —SO₂R³ or —SO₂NR³R⁴.

As heterocycloalkyls, there can be mentioned, e.g.: oxiranyl, oxethanyl, aziridinyl, azetidinyl, tetrahydrofuranyl, pyrrolidinyl, dioxolanyl, imidazolidinyl, pyrazolidinyl, dioxanyl, piperidinyl, morpholinyl, dithianyl, thimorpholinyl, piperazinyl, trithianyl, quinuclidinyl, pyrolidonyl, N-methylpyrolidinyl, 2-hydroxymethylpyrolidinyl, 3-hydroxypyrolidinyl, N-methylpiperazinyl, N-acetylpiperazinyl, N-methylsulfonylpiperazinyl, 4-hydroxypiperidinyl, 4-aminocarbonylpiperidinyl, 2-hydroxyethylpiperidinyl, 4-hydroxymethylpiperidinyl, nortropynyl, 1,1-dioxo-thiomorpholinyl, etc.

The term “cycloalkyl” is defined as a monocyclic alkyl ring, such as cyclopropyl, cyclobutyl, cyclopentyl, cyclohexyl or cycloheptyl, but also bicyclic rings or tricyclic rings, such as, for example, adamantanyl. The cycloalkyl can optionally also be benzocondensed, such as, e.g., (tetralin)yl, etc.

The term “halogen” is defined in each case as fluorine, chlorine, bromine or iodine.

As used herein, the term “aryl” is defined in each case as having 3 to 12 carbon atoms, preferably 6 to 12 carbon atoms, such as, for example, cyclopropenyl, cyclopentadienyl, phenyl, tropyl, cyclooctadienyl, indenyl, naphthyl, azulenyl, biphenyl, fluorenyl, anthracenyl etc, phenyl being preferred.

As used herein, the term “heteroaryl” is understood as meaning an aromatic ring system which comprises 3 to 16 ring atoms, preferably 5 or 6 or 9 or 10 atoms, and which contains at least one heteroatom which may be identical or different, said heteroatom being such as oxygen, nitrogen or sulfur, and can be monocyclic, bicyclic, or tricyclic, and in addition in each case can be benzocondensed. Preferably, heteroaryl is selected from thienyl, furanyl, pyrrolidinyl, pyrrotyl, oxazolyl, thiazolyl, imidazolyl, pyrazolyl, isoxazolyl, isothiazolyl, oxadiazolyl, triazolyl, thiadiazolyl, thia-4H-pyrazotyl etc., and benzo derivatives thereof, such as, e.g., benzofuranyl, benzothienyl, benzoxazolyl, benzimidazolyl, benzotriazolyl, indazolyl, indolyl, isoindolyl, etc.; or pyridyl, pyridazinyl, pyrimidinyl, pyrazinyl, triazinyl, etc., and benzo derivatives thereof, such as, for example, quinolinyl, isoquinolinyl, etc.; or oxepinyl, azocinyl, indolizinyl, indolyl, indolinyl, isoindolyl, indazolyl, benzimidazolyl, purinyl, etc., and benzo derivatives thereof; or quinolinyl, isoquinotinyl, phthalazinyl, quinazolinyl, quinoxatinyl, naphthpyridinyl, pteridinyl, carbazolyl, acridinyl, phenazinyl, phenothiazinyl, phenoxazinyl, xanthenyl, or oxepinyl, etc.

Preferred heteroaryl radicals are, for example, 5-membered ring heterocycles, such as thiophene, furanyl, oxazolyl, thiazole, imidazolyl and benzo derivatives thereof, and 6-membered ring heterocycles, such as pyridinyl, pyrimidinyl, triazinyl, quinotinyl, isoquinolinyl and benzo derivatives thereof.

As used herein, the term “C₁-C₆”, as used throughout this text, e.g. in the context of the definition of “C₁-C₆-alkyl”, “C₁-C₆-alkoxy”, “C-C₆-hydroxyalkyl”, “C₁-C₆-hydroxyalkoxy”, or “C₁-C₆-alkoxyaLkoxy”, etc., is to be understood as meaning an alkyl group having a finite number of carbon atoms of 1 to 6, i.e. 1, 2, 3, 4, 5, or 6 carbon atoms. It is to be understood further that said term “C₁-C₆” is to be interpreted as any sub-range comprised therein, e.g. C₁-C₆, C₂-C₅, C₃-C₄, C₁-C₂, C₁-C₃, C₁-C₄, C₁-C₅C₁-C₆; preferably C₁-C₂, C₁-C₃, C₁-C₄, C₁-C₅, C₁-C₆; more preferably C₁-C₄. In particular, as used herein, in the case of “alkenyl” or “alkynyl”, as used throughout this text, is to be understood as meaning an alkenyl or alkynyl group having a finite number of carbon atoms of 2 to 6, i.e. 2, 3, 4, 5, or 6 carbon atoms. It is to be understood further that said term “C₂-C₆” is to be interpreted as any subrange comprised therein, e.g. C₂-C₈, C₂-C₇, C₂-C₆, C₃-C₅, C₃-C₄, C₂-C₃, C₂-C₄, C₂-C₅; preferably C₂-C₃.

As used herein, the term “C₁-C₄”, as used throughout this text, e.g. in the context of the definition of “C₁-C₄-alkyl”, etc., is to be understood as meaning an alkyl group having a finite number of carbon atoms of 1 to 4, i.e. 1, 2, 3, or 4 carbon atoms. It is to be understood further that said term “C₁-C₄” is to be interpreted as any preferable sub-range comprised therein, e.g. C₁-C₄, C₂-C₃, C₁-C₂, C₁-C₃, C₂-C₄.

As used herein, the term “C₃-C₆”, as used throughout this text, e.g. in the context of the definitions of “C₃-C₆-cycloalkyl” or “C₃-C₆-heterocycloalkyl”, is to be understood as meaning a cyctoalkyl group having a finite number of carbon atoms, or a heterocycloaLkyl group having a finite number of ring atoms, of 3 to 6, i.e. 3, 4, 5, or 6 carbon atoms, preferably 5 or 6 carbon atoms. It is to be understood further that said term “C₃-C₆” is to be interpreted as any sub-range comprised therein, e.g. C₃-C₆ C₄-C₅, C₅-C₆; preferably C₅-C₆.

Isomers are defined as chemical compounds of the same summation formula but different chemical structure. In general, constitutional isomers and stereoisomers are distinguished.

Constitutional isomers have the same summation formula but are distinguished by the way in which their atoms or atom groups are linked. These include functional isomers, position isomers, tautomers or valence isomers.

Stereoisomers have basically the same structure (constitutional)—and thus also the same summation formula—but are distinguished by the spatial arrangement of the atoms.

In general, configurational isomers and conformational isomers are distinguished. Configurational isomers are stereoisomers that can be converted into one another only by bond breaking. These include enantiomers, diastereomers and E/Z (cis/trans)isomers.

Enantiomers are stereoisomers that behave Like image and mirror image to one another and do not exhibit any plane of symmetry. All stereoisomers that are not enantiomers are referred to as diastereomers. E/Z (cis/trans)isomers on double bonds are a special case.

Conformational isomers are stereoisomers that can be converted into one another by the rotation of single bonds.

To delimit types of isomerism from one another, see also the IUPAC Rules, Section E (Pure Appl. Chem. 45, 11-30, 1976).

The compounds of general formula I according to the invention also contain the possible tautomeric forms and comprise the E- or Z-isomers or, if a chiral center is present, also the racemates and enantiomers. Among the latter, double-bond isomers are also defined.

The compounds according to the invention can also be present in the form of solvates, especially hydrates, whereby the compounds according to the invention consequently contain polar solvents, especially water, as structural elements of the crystal lattice of the compounds according to the invention. The proportion of polar solvent, especially water, can be present in a stoichiometric or else unstoichiometric ratio. In the case of stoichiometric solvates and hydrates, hemi-, (semi-), mono-, sesqui-, di-, tri-, tetra-, penta-, etc., solvates or hydrates are also mentioned.

If an acid group is included, the physiologically compatible salts of organic and inorganic bases are suitable as salts, such as, for example, the readily soluble alkali and alkaline-earth salts, as well as N-methyl-glucamine, dimethyl-glucamine, ethyl-glucamine, lysine, 1,6-hexadiamine, ethanolamine, glucosamine, sarcosine, serinol, tris-hydroxy-methyl-amino-methane, aminopropane diol, Sovak base, and 1-amino-2,3,4-butanetriol.

If a basic group is included, the physiologically compatible salts of organic and inorganic acids are suitable, such as hydrochloric acid, hydrobromic acid, sulfuric acid, phosphoric acid, citric acid, tartaric acid, succinic acid, methylsulphonic acid, para-toluenesulphonic acid, etc:

Those compounds of general formula I in which
Q stands for phenyl,
A and B, independently of one another, stand for hydrogen, halogen, hydroxy, —NR³R⁴, cyano or nitro,
   or
   for C₁-C₄-alkyl, C₁-C₆-alkoxy or C₃-C₆-heterocycloalkyl that optionally is substituted in one or more places, in the same way or differently, with halogen, hydroxy, C₃-C₆-heterocycloalkyl or with the group —NR³R⁴ or —CO(NR³)-M, whereby the heterocycloalkyl itself optionally can be interrupted by one or more nitrogen, oxygen and/or sulfur atoms, and/or optionally can be interrupted by one or more —C(O)— or —SO₂ groups in the ring and/or optionally one or more double bonds can be contained in the ring and/or the ring itself optionally can be substituted in one or more places, in the same way or differently, with C₁-C₆-alkyl, C₃-C₆-cycloalkyl, C₁-C₆-hydroxyalkyl or with the group —NR³R⁴,
   or
   for —NR³C(O)-L, —NR³C(O)—NR³-L, —COR⁶, —O—(CH₂)ₚR⁶, —CO(NR³)-M, —NR³(CS)NR³R⁴, —NR³SO₂-M, —SO₂—NR³R⁴, —SO₂(NR³)-M or —O—(CH₂)ₚaryl,
p stands for an integer of 0, 1, 2, 3, or 4,
L stands for C₁-C₆-alkyl or C₃-C₆-heterocycloalkyl that optionally is substituted in one or more places, in the same way or differently, with C₁-C₆-hydroxyalkoxy, C₁-C₆-alkoxyalkoxy, C₃-C₆-heterocycloalkyl or with the group —NR³R⁴,
   whereby the heterocycloalkyl itself optionally can be interrupted by one or more nitrogen, oxygen and/or sulfur atoms and/or optionally can be interrupted by one or more —C(O)—or —SO₂ groups in the ring and/or optionally one or more double bonds can be contained in the ring and/or the ring itself optionally can be substituted in one or more places, in the same way or differently, with C₁-C₆-alkyl, C₃-C₆-cycloalkyl, C₁-C₆-hydroxyalkyl or with the group —NR³R⁴,
M stands for C₁-C₆-alkyl that optionally is substituted in one or more places, in the same way or differently, with the group —NR³R⁴ or C₃-C₆-heterocycloalkyl,
X stands for —NH— or —NR⁵—,
R¹ stands for C₁-C₄-alkyl, C₃-cycloalkyl, allyl or propargyl that optionally is substituted in one or more places, in the same way or differently, with halogen,
R² stands for hydrogen or for C₁-C₆-alkyl, C₁-C₆-alkoxy, C₁-C₆-alkenyl, C₁-C₆-alkynyl, C₃-C₆-cycloalkyl, C₃-C₆-heterocycloalkyl, aryl or heteroaryl that optionally is substituted in one or more places, in the same way or differently, with halogen, hydroxy, cyano, C₁-C₆-alkyl, C₁-C₆-alkoxy, C₁-C₆-hydroxyalkyl, C₃-C₆-cycloalkyl, C₃-C₆-heterocycloalkyl, C₁-C₆-alkynyl, aryl, aryloxy, heteroaryl or with the group —S—C₁-C₆-alkyl, —COR⁶, —NR³R⁴, —NR³C(O)-L or —NR³COOR⁷,
   whereby the heterocycloalkyl itself optionally can be interrupted by one or more nitrogen, oxygen and/or sulfur atoms and/or optionally can be interrupted by one or more —C(O)— or —SO₂ groups in the ring and/or optionally one or more double bonds can be contained in the ring,
   and whereby aryl, heteroaryl, C₃-C₆-cycloalkyl and/or the C₃-C₆-heterocycloalkyl ring in each case itself optionally can be substituted in one or more places, in the same way or differently, with cyano, halogen, hydroxy, C₁-C₆-alkyl, C₁-C₆-hydroxyalkyl; C₁-C₆-alkoxy, C3-C₆-cycloalkyl, C₃-C₆-heterocycloalkyl, aryl, benzyl or heteroaryl that optionally is substituted in one or more places, in the same way or differently, with halogen, ’or
   for the group —NR³R⁴, —NR³C(O)-L, —NR³(CS)NR ³R⁴,
   or
R² and R⁵ together form a C₃-C₆-heterocycloalkyl ring, which is interrupted at least once by nitrogen and optionally can be interrupted in one or more places by oxygen or sulfur and/or optionally can be interrupted by one or more —C(O)— or —SO₂— groups in the ring and/or optionally one or more double bonds can be contained in the ring,
   and/or the ring itself optionally can be substituted in one or more places, in the same way or differently, with cyano, halogen, hydroxy, C₁-C₆-alkyl, C₃-C₆-cycloalkyl, C₁-C₆-hydroxyalkyl, C₁-C₆-alkoxyalkyl or with the group —NR³R⁴ or —COR⁶ and/or can be substituted with aryl or heteroaryl that optionally is substituted in one or more places, in the same way or differently, with halogen, C₁-C₆-alkoxy or with the group —COR⁶,
R³ and R⁴, independently of one another, stand for hydrogen or for C₁-C₆-alkyl, C₁-C₆-alkoxy, —CO—C₁-C₆-alkyl or aryl that optionally is substituted in one or more places, in the same way or differently, with halogen, hydroxy, C₃-C₆-heterocycloalkyl, C₁-C₆-hydroxyalkoxy or with the group —NR³R⁴,
   whereby the heterocycloalkyl itself optionally can be interrupted by one or more nitrogen, oxygen and/or sulfur atoms and/or optionally can be interrupted by one or more —C(O)— or —SO₂— groups in the ring and/or optionally one or more double bonds can be contained in the ring and whereby the C₃-C₆-heterocycloalkyl ring itself in each case optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl or with the group —NR³R⁴ or —CO—NR³R⁴,
   or
R³ and R⁴ together form a C₃-C₆-heterocycloalkyl ring, which is interrupted at least once by nitrogen and optionally can be interrupted in one or more places by oxygen or sulfur and/or optionally can be interrupted by one or more —C(O)— or —SO₂— groups in the ring and/or optionally one or more double bonds can be contained in the ring
   and/or the heterocycloalkyl ring itself optionally can be substituted in one or more places, in the same way or differently, with C₁-C₆-alkyl, C₃-C₆-cycloalkyl, C₁-C₆-hydroxyalkyl, C₁-C₆-alkoxyalkyl, cyano, hydroxy or with the group —NR³R⁴,
R⁵ stands for C₁-C₆-alkyl, C₁-C₆-alkenyl or C₁-C₆-alkynyl that optionally is substituted in one or more places, in the same way or differently, with halogen, hydroxy, cyano, C₁-C₆-alkoxy, C₃-C₆-cycloalkyl, C₃-C₆-heterocycloalkyl or with the group —NR³R⁴, whereby the heterocycloalkyl itself optionally can be interrupted by one or more nitrogen, oxygen and/or sulfur atoms and/or optionally can be interrupted by one or more —C(O)— or —SO₂— groups in the ring and/or optionally one or more double bonds can be contained in the ring and
   whereby the C₃-C₆-heterocycloalkyl ring itself in each case optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group —NR³R⁴ or —CO—NR³R⁴,
R⁶ stands for hydroxy, C₁-C₆-alkyl, C₁-C₆-alkoxy or the group —NR³R⁴,
R⁷ stands for —(CH₂)ₙ-aryl or —(CH₂)ₙ-heteroaryl, and
n stands for an integer of 1, 2, 3, 4, 5 or 6,
   as well as the solvates, hydrates, stereoisomers, diastereomers, enantiomers and salts thereof,
   have been shown to be especially effective.

Those compounds of general formula 1, in which
Q stands for phenyl,
A and B, independently of one another, stand for hydrogen, halogen, or for C₁-C₄-alkyl or pyrrolidinyl that optionally is substituted in one or more places, in the same way or differently, with halogen, hydroxy or with the group —NR³R⁴ or —CO(NR³)-M,
M stands for C₁-C₆-alkyl that optionally is substituted in one or more places, in the same way or differently, with the group —NR³R⁴ or C₃-C₆-heterocycloalkyl,
X stands for —NH—,
R¹ stands for C₁-C₄-alkyl that optionally is substituted in one or more places, in the same way or differently, with halogen,
R² stands for hydrogen or for C₁-C₆-alkyl or C₁-C₆-alkynyl that optionally is substituted in one or more places, in the same way or differently, with halogen, cyano, or C₁-C₆-alkoxy,
R³ and R⁴, independently of one another, stand for hydrogen or for C₁-C₆-alkyl, C₁-C₆-alkoxy, —CO—C₁-C₆-alkyl or aryl that optionally is substituted in one or more places, in the same way or differently, with halogen, hydroxy, C₃-C₆-heterocycloalkyl, C₁-C₆-hydroxyalkoxy or with the group —NR³R⁴,
   whereby the heterocycloalkyl itself optionally can be interrupted by one or more nitrogen, oxygen and/or sulfur atoms, and/or optionally can be interrupted by one or more —C(O)— or —SO₂— groups in the ring and/or optionally one or more double bonds can be contained in the ring, and
   whereby the C₃-C₆-heterocycloalkyl ring itself optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group —NR³R⁴ or —CO—NR³R⁴,
   or
R³ and R⁴ together form a C₃-C₆-heterocycloalkyl ring, which is interrupted at least once by nitrogen and optionally can be interrupted in one or more places by oxygen or sulfur and/or optionally can be interrupted by one or more —C(O)— or —SO₂— groups in the ring and/or optionally one or more double bonds can be contained in the ring,
   and/or the heterocycloalkyl ring itself optionally can be substituted in one or more places, in the same way or differently, with C₁-C₆-alkyl, C₃-C₆-cycloalkyl, C₁-C₆-hydroxyalkyl, C₁-C₆-alkoxyalkyl, cyano, hydroxy or with the group —NR³R⁴, and
R⁶ stands for hydroxy or C₁-C₆-alkoxy,
   as well as the solvates, hydrates, stereoisomers, diastereomers, enantiomers and salts thereof,
   are quite especially effective.

Those compounds of general formula I, in which
Q stands for phenyl,
A and B, independently of one another, stand for hydrogen, halogen, hydroxy, methoxy or pyrrolidinyl,
X stands for —NH—,
R¹ stands for ethyl,
R² stands for ethyl or propynyl,
   as well as the solvates, hydrates, stereoisomers, diastereomers, enantiomers and salts thereof,
   are extremely effective.

A subject of this invention is also the use of the compounds of general formula I, which may be for the production of a pharmaceutical agent for treating cancer, auto-immune diseases, chemotherapy agent-induced alopecia and mucositis, cardiovascular diseases, infectious diseases, nephrological diseases, chronic and acute neurodegenerative diseases and viral infections.

A subject of this invention is also the use of the compounds of general formula I for the production of a pharmaceutical agent for treating cancer, solid tumours and leukemia; auto-immune diseases: psoriasis, alopecia and multiple sclerosis; cardiovascular diseases: stenoses, arterioscleroses, and restenoses; infectious diseases: diseases that are caused by unicellular parasites; nephrological diseases: glomerulonephritis; chronic neurodegenerative diseases: Huntington's disease, amyotrophic lateral sclerosis, Parkinson's disease, AIDS dementia and Alzheimer's disease; acute neurodegenerative diseases: ischemias of the brain and neurotraumas; and viral infections: cylomegalic infections, herpes, hepatitis B and C, and HIV.

The compounds according to the invention can be used in the case of cancer, autoimmune diseases, cardiovascular diseases, infectious diseases, nephrological diseases, neurodegenerative diseases and viral infections.

The invention also comprises pharmaceutical agents that contain at least one compound of general formula I.

Such pharmaceutical agents are used in the treatment of cancer, autoimmune diseases, cardiovascular diseases, infectious diseases, nephrological diseases, neurodegenerative diseases and viral infections.

In general, the compounds according to the invention are mixed in the pharmaceutical agents with suitable formulation substances and vehicles.

A subject of this invention is thus also a pharmaceutical preparation for enteral, parenteral and oral administration.

To use the compounds of formula I as pharmaceutical agents, the tatter are brought into the form of a pharmaceutical preparation, which, in addition to the active ingredient for the enteral or parenteral administration, contains suitable pharmaceutical, organic or inorganic inert carrier materials, such as, for example, water, gelatin, gum Arabic, lactose, starch, magnesium stearate, talc, plant oils, polyatkylene glycols, etc. The pharmaceutical preparations can be present in solid form, for example as tablets, coated tablets, suppositories, or capsules, or in liquid form, for example as solutions, suspensions or emulsions. Moreover, they optionally contain adjuvants such as preservatives, stabilizing agents, wetting agents or emulsifiers, salts for changing the osmotic pressure, or buffers.

For parenteral administration, in particular injection solutions or suspensions, in particular aqueous solutions of the active compounds in polyhydroxyethoxylated castor oil, are suitable.

As carrier systems, surface-active adjuvants such as salts of bile acids or animal or plant phospholipids, but also mixtures thereof as well as liposomes or components thereof can also be used.

For oral administration, in particular tablets, coated tablets or capsules with talc and/or hydrocarbon vehicles or binders, such as, for example, lactose, corn or potato starch, are suitable. The administration can also be done in liquid form, such as, for example, as a juice, to which optionally a sweetener, or, if necessary, one or more flavoring substances, is added.

The dosage of the active ingredients can vary depending on the method of administration, age and weight of the patient, type and severity of the disease to be treated and similar factors. The daily dose is 0.5-1,000 mg, preferably 50-200 mg, whereby the dose can be given as a single dose to be administered once or divided into two or more daily doses.

The above-described formulations and dispensing forms are also subjects of this invention.

In particular, the compounds according to the invention are used as inhibitors of polo-like kinases. Polo-like kinases are defined as in particular Plk 1, Plk 2, Plk 3 and Plk 4.
General Production Diagram for Producing the Compounds According to the Invention

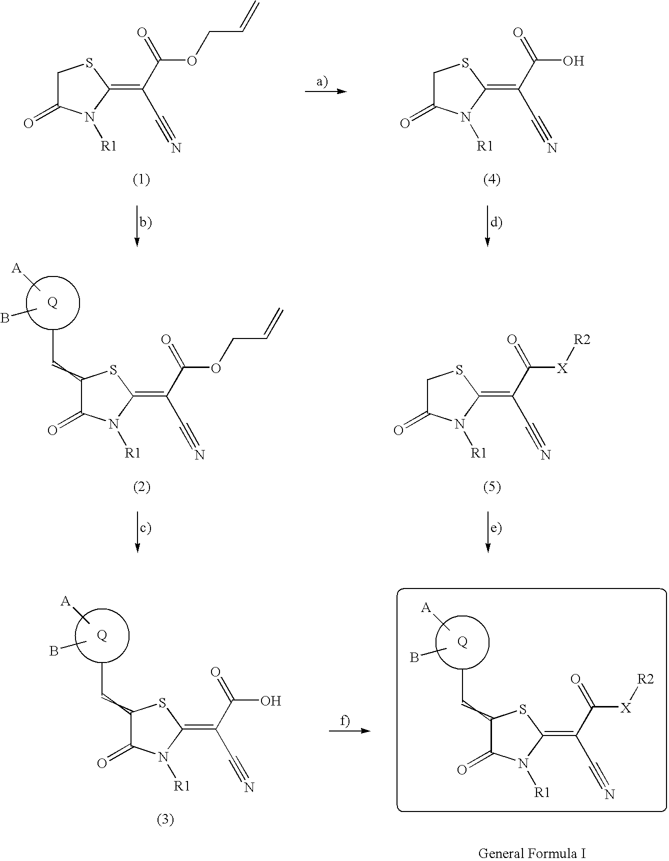

Reaction conditions: a) Saponification in the presence of Pd-tetrakis-triphenylphosphine and barbituric acid; b) Condensation with aldehydes; c) Saponification in the presence of Pd-tetrakis-triphenylphosphine and barbituric acid; d) Amide formation from the free carboxylic acid; e) Condensation with aldehydes; f) Amide formation from the free carboxylic acid.

The production of the compounds of general formula I can be carried out in principle via two alternative synthesis routes. The process variant I comprises the intermediate products 2 and 3 starting from the starting material 1 that is already described in the International Application WO 03/093249. The process variant II comprises the intermediate products 4 and 5 starting from the same starting material 1. Both process variants are also suitable for use in parallel-synthetic production processes of compounds of general formula I. Based on the process, the radicals X—R2 or Q of the test compounds according to the invention can be widely varied in the last synthesis stage in each case.

In the process variant I, the formation of a by-product 6 was observed in the reaction of the intermediate product 2 to the intermediate product 3. In this connection, in addition to the systematic saponification of the allyl ester functionality, surprisingly enough, an additional decarboxylation takes place.

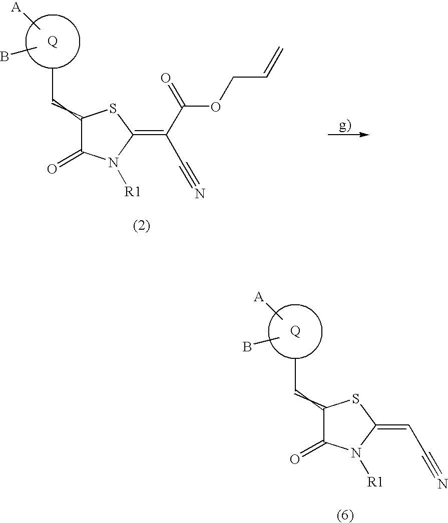

Reaction conditions: g) Saponification in the presence of Pd-tetrakis-triphenylphosphine and barbituric acid at elevated temperature.

1. Production of the Intermediate Products of Formula (2) According to the Invention

Intermediate Product (ZP1)

### 5-[1-(4-Bromo-phenyl)-meth-(Z)-ylidene]-3-ethyl-4-oxo-thiazolidin-(2Z)-ylidene]-cyano-acetic acid allyl ester

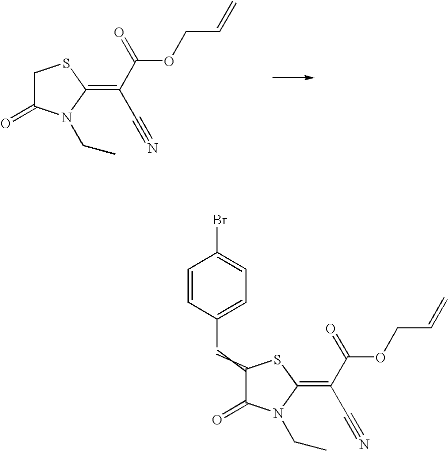

1.01 g of the starting material (4.0 mmol) that is described in Patent Application PCT/EP2004/012242 A is dissolved in 10 ml of tetrahydrofuran, mixed with 740 mg (4.0 mmol) of p-bromobenzaldehyde and 0.04 ml of piperidine, and stirred for 48 hours at room temperature. The reaction mixture is then concentrated by evaporation almost until the drying is completed and purified without further working-up by crystallization from ethyl acetate. 938 mg (56%) of the title compound is obtained as a pH-dependent (E/Z)-isomer mixture.

1H-NMR (DMSO-d6, stored with K₂CO₃, main isomer): δ=1.30 (t, 3H); 4.28 (q, 2H); 4.77 (m, 2H); 5.31 (m, 1H); 5.41 (m, 1H); 6.01 (m, 1H); 7.66 (d, 2H); 7.82 (d, 2H); 7.88 (s, 1H) ppm.

Similarly produced are also:

TABLE 1
Aldehyde Condensates:

| | | | Molecular |
|---|---|---|---|
| | | | Weight/ |
| Example | Structure and Name of the Main | | MS (ESI) |
| No. | Isomer | ¹H-NMR | [M + 1]⁺ |
| ZP2 | 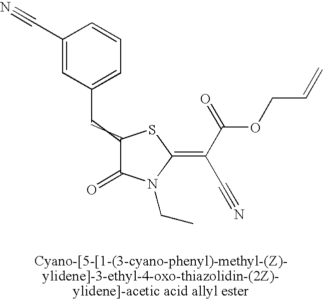 | (DMSO-d6, Stored with K₂CO₃, Main Isomer): δ =1.32(t, 3H); 4.28(q, 2H); 4.78(m, 2H); 5.30(dd, 1H); 5.41(dd, 1H); 5.9-6.1(m, 1H); 7.82(m, 1H); 7.92(s, 1H); 7.98(d, 1H); 8.18(s, 1H) ppm. | MW: 341.39 MS (ESI) [M + 1]⁺: 342 |
| ZP3 | 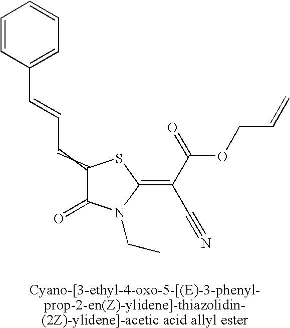 | (DMSO-d6, Stored with K₂CO₃, Main Isomer): δ =1.28(t, 3H); 4.28(q, 2H); 4.77(m, 2H); 5.29(dd, 1H); 5.40(dd, 1H); 5.9-6.1(m, 1H); 7.29(d, 1H); 7.36-7.48(m, 5H); 7.63(d, 1H); 7.74(m, 1H) ppm. | MW: 341.39 MS (ESI) [M + 1]⁺: 342 |
| ZP4 | 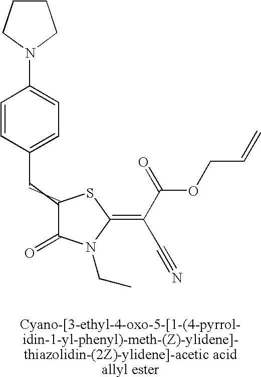 | (DMSO-d6, Stored with K₂CO₃, Main Isomer): δ =1.28(t, 3H); 2.00(m, 4H); 3.31(m, 4H); 4.26(q, 2H); 4.77(m, 2H); 5.30(dd, 1H); 5.41(dd, 1H); 5.9-6.1(m, 1H); 6.70(d, 2H); 7.52(d, 2H); 7.72(m, 1H) ppm. | MW: 343.41 MS (ESI) [M + 1]⁺: 344 |
| ZP5 | 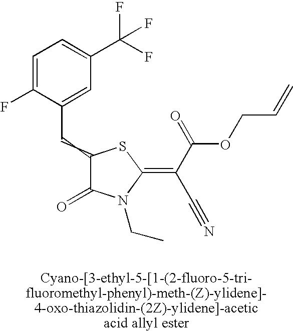 | (DMSO-d6, Stored with K₂CO₃, Main Isomer): δ =1.32(t, 3H); 4.30(q, 2H); 4.78(m, 2H); 5.29(dd, 1H); 5.41(dd, 1H); 5.9-6.1(m, 1H); 7.68(m, 1H); 7.87(s, 1H); 7.94-8.04(m, 2H) ppm. | |
| ZP6 | 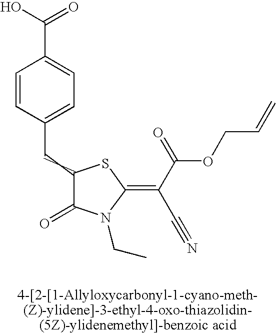 | (DMSO-d6, Stored with K₂CO₃, Main Isomer): δ =1.32(t, 3H); 3.60(br. s, 1H); 4.30(q, 2H); 4.78(m, 2H); 5.30(dd, 1H); 5.41(dd, 1H); 5.9-6.1(m, 1H); 7.76(d, 2H); 7.93(s, 1H); 8.08(d, 1H) ppm. | |
| ZP7 | 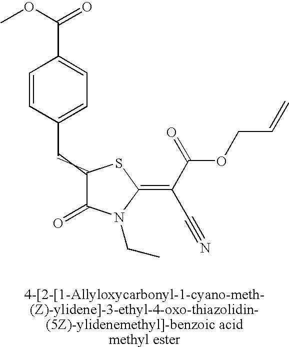 | (DMSO-d6, Stored with K₂CO₃, Main Isomer): δ =1.36(t, 3H); 3.93(s, 3H); 4.35(q, 2H); 4.8(m, 2H); 5.31(dd, 1H); 5.42(dd, 1H); 5.9-6.1(m, 1H); 7.82(d, 2H); 7.92(s, 1H); 8.12(d, 1H) ppm. | |
| ZP8 | 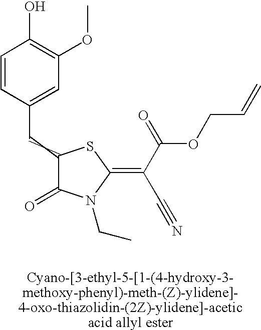 | (DMSO-d6, Stored with K₂CO₃, Main Isomer): δ =1.30(t, 3H); 3.84(s, 3H); 4.28(q, 2H); 4.78(m, 2H); 5.29(dd, 1H); 5.41(dd, 1H); 5.9-6.1(m, 1H); 6.98(d, 1H); 7.22(dd, 2H); 7.32(d, 1H); 7.82(s, 1H) ppm. | |
| ZP9 | 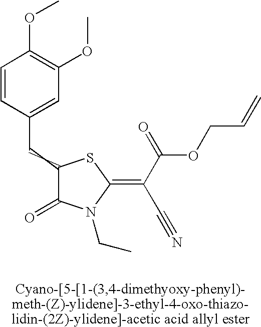 | (DMSO-d6, Stored with K₂CO₃, Main Isomer): δ =1.34(t, 3H); 3.86(s, 3H); 3.90(s, 3H); 4.34(q, 2H); 4.79(m, 2H); 5.29(dd, 1H); 5.42(dd, 1H); 5.9-6.1(m, 1H); 7.20(d, 2H); 7.31(s, 1H); 7.32(d, 1H); 7.83(s, 1H) ppm. | |
| ZP10 | 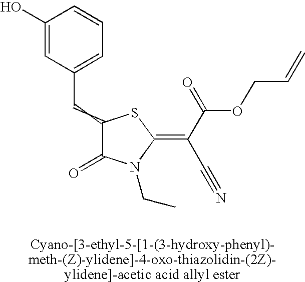 | (DMSO-d6, Stored with K₂CO₃, Main Isomer): δ =1.29(t, 3H); 4.28(q, 2H); 4.78(m, 2H); 5.30(dd, 1H); 5.41(dd, 1H); 5.9-6.1(m, 1H); 6.93(dd, 1H); 7.09(m, 2H); 7.14(d, 1H); 7.38(dd, 1H); 7.79(s, 1H); 9.8-10.1(br. s, 1H) ppm. | |
| ZP11 | 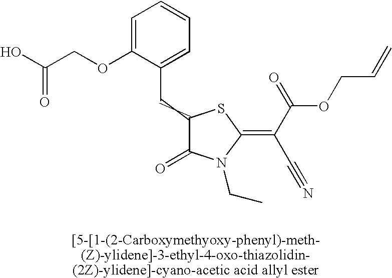 | (DMSO-d6, Stored with K₂CO₃, Main Isomer): δ =1.31(t, 3H); 4.28(q, 2H); 4.72(s, 2H); 4.76(m, 2H); 5.28(dd, 1H); 5.9-6.1(m, 1H); 7.04(d, 1H); 7.16(m, 1H); 7.48(m, 1H); 7.55(d, 1H); 8.17(s, 1H) ppm. | |
| ZP12 | 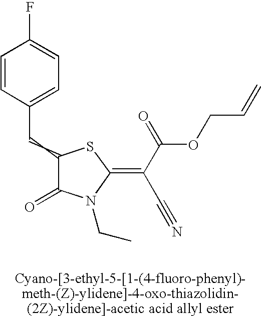 | (DMSO-d6, Stored with K₂CO₃, Main Isomer): δ =1.30(t, 3H); 4.29(q, 2H); 4.77(m, 2H); 5.29(dd, 1H); 5.41(dd, 1H); 5.9-6.1(m, 1H); 7.45(m, 2H); 7.78(m, 2H); 7.92(s, 1H) ppm. | |
| ZP13 | 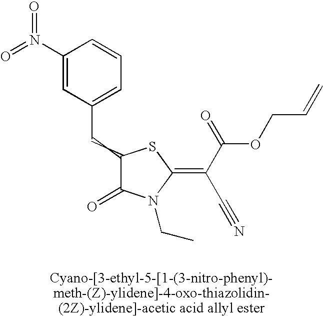 | (DMSO-d6, Stored with K₂CO₃, Main Isomer): δ =1.32(t, 3H); 4.31(q, 2H); 4.79(m, 2H); 5.30(dd, 1H); 5.41(dd, 1H); 5.9-6.1(m, 1H); 7.90(m, 1H); 8.07(s, 1H); 8.12(d, 1H); 8.34(m, 1H); 8.56(m, 1H) ppm. | |

2. Production of the Intermediate Products of the Formula According to the Invention

Intermediate Product (ZP14)

### Cyano-[3-ethyl-4-oxo-5-[1-(4-pyrrolidin-1-yl-phenyl)-meth-(Z)-ylidene]-thiazolidin-(2Z)-ylidene]-acetic Acid

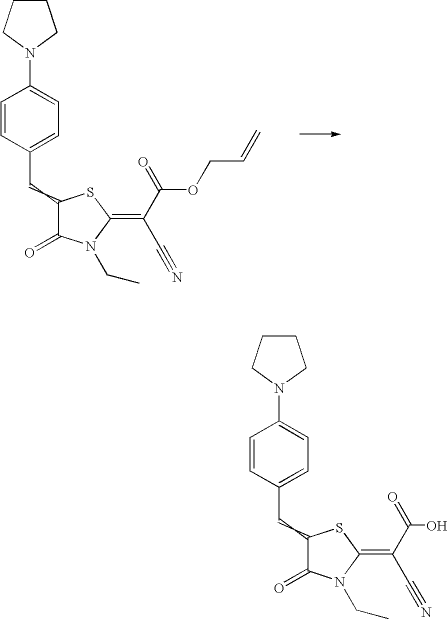

1.0 g (about 2.44 mmol) of cyano-[3-ethyl-4-oxo-5-[1-(4-pyrrolidin-1-yl-phenyl)-meth-(Z)-ylidene]-thiazolidin-(2Z)-ylidene]-acetic acid allyl ester is stirred together with 341 mg (2.66 mmol) of barbituric acid and 277 mg (0.24 mmol) of palladium-tetrakis-triphenylphosphine in 10 ml of tetrahydrofuran for 24 hours at room temperature. For working-up, the crude product is mixed with ethyl acetate, and the precipitate that is formed is suctioned off. The thus isolated product (648 mg, 71%) is used without further purification in the next steps.

1H-NMR (DMSO-d6, stored with K₂CO₃, main isomer): δ=1.25 (t, 3H); 1.97 (m, 4H); 3.38 (m, 4H); 4.27 (q, 2H); 6.72 (m, 2H), 7.5-7.65 (m, 2H), 7.71 (s, 1H) ppm.

Production of the Intermediate Products of Formulae (4) and (5) According to Process Variant II

ZP15

### 2-Cyano-2-[3-ethyl-4-oxo-3-yl-meth-(Z)-ylidene]-thiazolidin-(2Z)-ylidene]-acetic acid

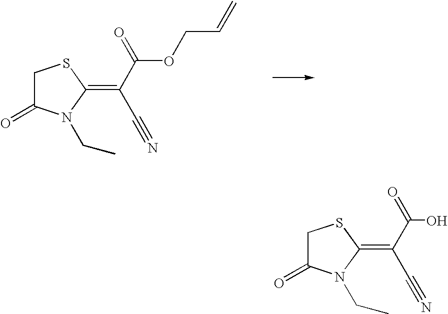

40 g (about 0.16 mol) of the allyl ester already described in Patent Application WO 03/093249 is stirred together with 22.18 g (˜0.17 mmol) of barbituric acid and 18.3 g (10 mol %) of palladium-tetrakis-triphenylphosphine in 50 ml of THF over a period of 72 hours at room temperature. After TLC monitoring of the reaction mixture, the solvent is removed in a vacuum. The crude product is used without further purification in the subsequent reactions and contains about 50% of the desired carboxylic acid.

An analytically pure sample was obtained by filtration and subsequent boiling-off of the filter cake with toluene.

1H-NMR (DMSO-d6, stored with K₂CO₃, main isomer): δ=1.20 (t, 3H); 3.60 (s, 2H); 4.12 (q, 2H); 11.1 (s, 1H) ppm.

ZP16

### 2-Cyano-2-[3-ethyl-4-oxo-3-yl-meth-(Z)-ylidene]-thiazolidin-(2Z)-ylidene]-N-ethyl-acetamide

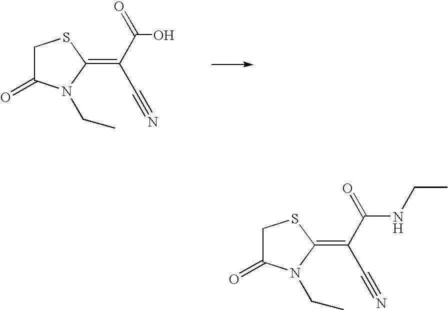

15 g of the crude product 2-cyano-2-[3-ethyl-4-oxo-3-yl-meth-(Z)-ylidene]-thiazolidin-(2Z)-ylidene]-acetic acid is introduced together with 21.2 ml of ethylamine and 11.8 g of sodium bicarbonate into 200 ml of DMF. After 30 minutes of stirring at room temperature, 13.8 g of TBTU is added, and the reaction mixture is further stirred overnight at room temperature. For working-up, the crude product is mixed with ethyl acetate. The aqueous phase is extracted twice more with 100 ml each of ethyl acetate. The combined organic phases are extracted in succession with saturated sodium bicarbonate solution and saturated sodium chloride solution. Then, the organic phase is dried on sodium sulfate, filtered, and concentrated by evaporation.

By crystallization from ethanol, 4.05 g (48% relative to the indicated content of 2-cyano-2-[3-ethyl-4-oxo-3-yl-meth-(Z)-ylidene]-thiazolidin-(2Z)-ylidene]-acetic acid in the crude product) of the desired product is isolated from the crude product.

1H-NMR (DMSO-d6, stored with K₂CO₃, main isomer): δ=1.00 (t, 3H); 1.16 (t, 3H); 3.14 (m, 2H); 3.77 (s, 2H); 4.05 (q, 2H); 7.74 (m, 1H) ppm.

Similarly produced are also:

TABLE 3a
Amides:

| | | | Molecular |
|---|---|---|---|
| | | | Weight/ |
| Example | | | MS (ESI) |
| No. | Structure and Name | ¹H-NMR | [M + 1]⁺ |
| ZP17 | 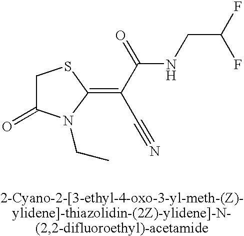 | (DMSO-d6, Stored with K₂CO₃): δ =1.17(t, 3H); 3.03(m, 2H); 3.78(s, 2H); 4.07(q, 2H); 6.02(m, 1H); 8.01(m, 1H) ppm. | |
| ZP18 | 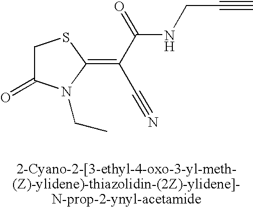 | (DMSO-d6, Stored with K₂CO₃): δ =1.16(t, 3H); 3.03(m, 2H); 3.79(s, 2H); 3.86(m, 2H); 4.06(q, 2H); 8.18(m, 1H) ppm. | |
| ZP19 | 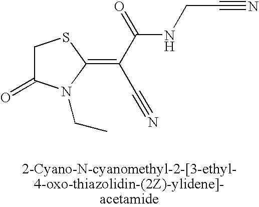 | (DMSO-d6, Stored with K₂CO₃): δ =1.35(t, 3H); 3.70(s, 2H); 4.24(m, 4H); 6.67(m, 1H) ppm. | |

3. Production of the End Products According to the Invention

### EXAMPLE 1

### 2-Cyano-N-ethyl-2-[3-ethyl-4-oxo-5-[1-(4-pyrrolidin-1-yl-phenyl)-meth-(Z)-ylidene]-thiazolidin-(2Z) -ylidene]-acetamide

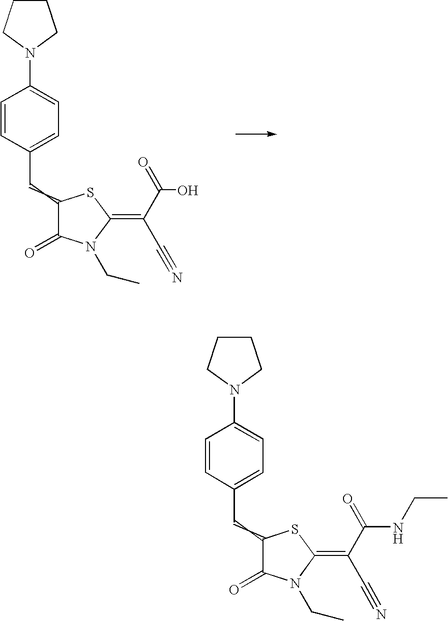

50 mg (0.14 mmol) of cyano-[3-ethyl-4-oxo-5-[1-(4-pyrrolidin-1-yl-phenyl)-meth-(Z)-ylidene]-thiazolidin-(2Z)-ylidene]-acetic acid is introduced together with 0.2 ml (0.41 mmol) of ethylamine (2 M in THF) and 154 mg (0.41 mmol) of HATU in 10 ml of DMF, and it is stirred overnight at room temperature. For working-up, the crude product is mixed with ethyl acetate and extracted 3 times with 10 ml each of water. The combined organic phases are dried on sodium sulfate, filtered and concentrated by evaporation. The purification of the crude product is carried out by column chromatography on silica gel. 32 mg (60%) of a yellow solid is isolated.

As an alternative to the production of amides via the free carboxylic acids, the corresponding compound can also be produced by condensation of the corresponding amides with aldehydes:

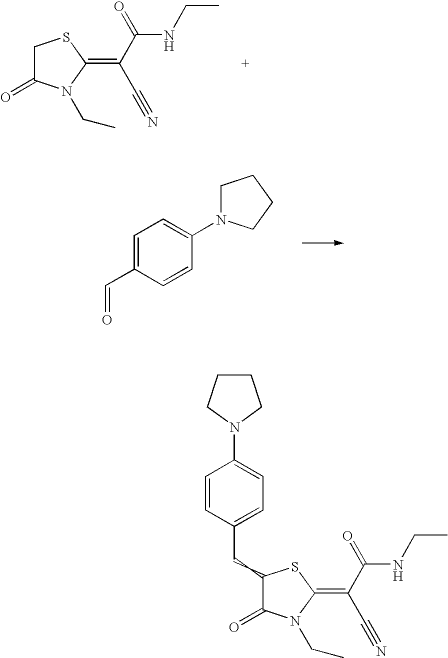

50 mg (0.21 mmol) of 2-cyano-2-[3-ethyl-4-oxo-3-yl-meth-(Z)-ylidene]-thiazolidin-(2Z)-ylidene]-N-ethyl-acetamide is stirred together with 53 mg (0.30 mmol) of 4-pyrrolidin-1-yl-benzaldehyde and 10 μl of piperidine in 10 ml of THF at room temperature overnight. After the reaction is completed, the desired product is filtered off; the filtrate is discarded. 54 mg (70%) of a yellow solid is isolated. As an alternative to this, the isolation of the desired product can be carried out by aqueous working-up with ethyl acetate, drying on sodium sulfate and subsequent purification of the crude product by column chromatography on silica gel.

1H-NMR (DMSO-d6, stored with K₂CO₃, main isomer; measurement made at 350 K): δ=1.13 (t, 3H); 1.29 (t, 3H); 2.02 (m, 4H); 3.25 (q, 2H); 3.36 (m, 4H); 4.29 (q, 2H); 6.72 (d, 2H); 7.52 (d, 2H); 7.57 (s, 1H); 7.62 (s, 1H) ppm.

Similarly produced are also:

TABLE 3
Amides:

| | | | Molecular |
|---|---|---|---|
| | | | Weight/ |
| Example | | | MS (ESI) |
| No. | Structure and Name | ¹H-NMR | [M + 1]⁺ |
| 2 | 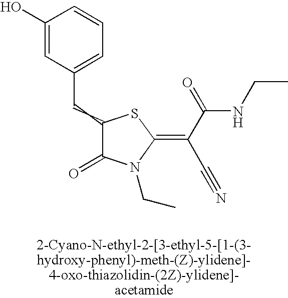 | (DMSO-d6, Stored with K₂CO₃, Main Isomer): δ =1.05(t, 3H); 1.25(t, 1H); 3.19(m, 2H); 4.22(q, 2H); 6.85(m, 1H); 7.06(s, 1H); 7.09(m, 1H); 7.34(m, 1H); 7.63(s, 1H); 8.06(m, 1H); 9.9(s, br, 1H) ppm. | MW: 343.41 MS (ES+) [M + 1]⁺: 344 |
| 3 | 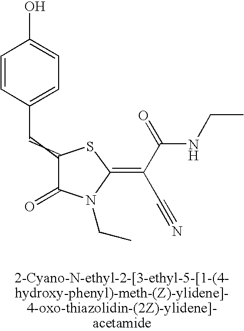 | (DMSO-d6, Stored with K₂CO₃, Main Isomer): δ =1.04(t, 3H); 1.23(t, 1H); 3.17(m, 2H); 4.21(q, 2H); 6.82(m, 2H); 6.98(m, 2H); 7.11(s, 1H); 7.93(m, 1H) ppm. | MW: 343.41 MS (ES+) [M + 1]⁺: 344 |
| 4 | 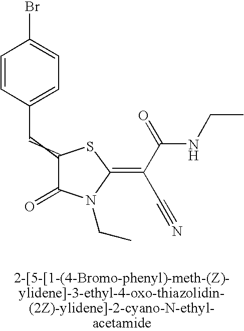 | (DMSO-d6, Stored with K₂CO₃, Main Isomer): δ =1.04(t, 3H); 1.26(t, 1H); 3.18(m, 2H); 4.23(q, 2H); 7.61(m, 2H); 7.69(s, 1H); 7.76(m, 2H); 8.08(m, 1H) ppm. | MW: 406.30 MS (ES+) [M + 1]⁺: 406/408 |
| 5 | 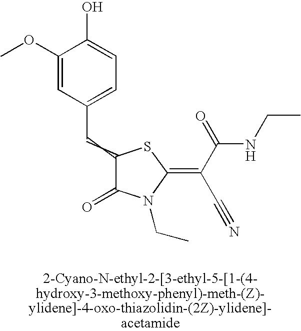 | (DMSO-d6, Stored with K₂CO₃, Main Isomer): δ =1.04(t, 3H); 1.23(t, 1H); 3.18(m, 2H); 3.76(s, 3H); 4.21(q, 2H); 6.73(m, 1H); 7.1(m, 2H); 7.60(s, 1H); 7.85(m, 1H) ppm. | MW: 373.43 MS (ES+) [M + 1]⁺: 374 |
| 6 | 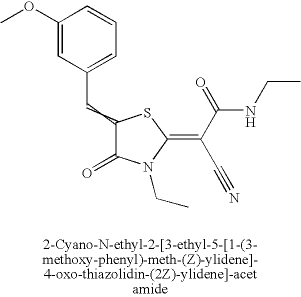 | (DMSO-d6, Stored with K₂CO₃, Main Isomer): δ =1.05(t, 3H); 1.27(t, 1H); 3.19(m, 2H); 3.78(s, 3H); 4.23(q, 2H); 7.05(m, 1H); 7.2(m, 2H); 7.48(m, 1H); 7.70(s, 1H); 8.04(m, 1H) ppm. | MW: 357.43 MS (ES+) [M + 1]⁺: 358 |
| 7 | 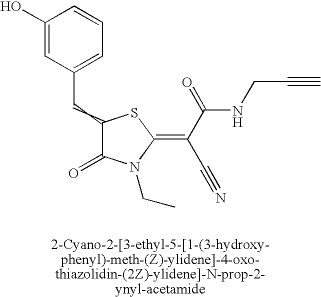 | (DMSO-d6, Stored with K₂CO₃, Main Isomer): δ =1.27(t, 1H); 3.05(m, 1H); 3.90(m, 2H); 4.23(q, 2H); 6.86(m, 1H); 7.06(s, 1H); 7.08(m, 1H); 7.32(m, 1H); 7.63(s, 1H); 8.43(m, 1H); 9.9(s, br, 1H) ppm. | MW: 343.41 MS (ES+) [M + 1]⁺: 344 |
| 8 | 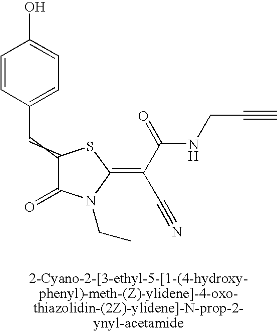 | (DMSO-d6, Stored with K₂CO₃, Main Isomer): δ =1.24(t, 1H); 3.06(m, 1H); 3.88(m, 2H); 4.23(q, 2H); 6.93(m, 2H); 7.54(m, 2H); 7.68(s, 1H); 8.48(m, 1H); 10.4(s, br, 1H) ppm. | MW: 353.41 MS (ES+) [M + 1]⁺: 354 |
| 9 | 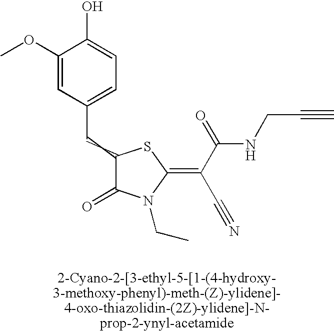 | (DMSO-d6, Stored with K₂CO₃, Main Isomer): δ =1.25(t, 1H); 3.07(m, 1H); 3.80(s, 3H); 3.89(m, 2H); 4.23(q, 2H); 6.96(m, 1H); 7.16(m, 1H); 7.27(m, 1H); 7.69(s, 1H); 8.38(m, 1H); 9.9(s, br, 1H) ppm. | |
| 10 | 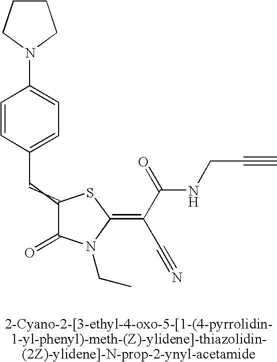 | (DMSO-d6, Stored with K₂CO₃, Main Isomer): δ =1.23(t, 1H); 1.92(m, 4H); 3.05(m, 1H); 3.29(m, 4H); 3.89(m, 2H); 4.22(q, 2H); 6.68(d, 2H); 7.45-7.60(m, 3H); 8.29(m, 1H) ppm. | |
| 11 | 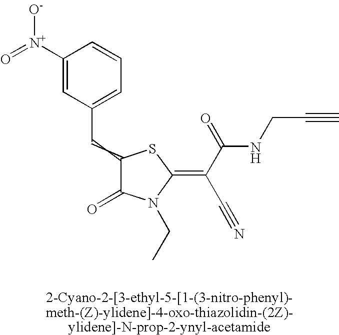 | (DMSO-d6, Stored with K₂CO₃, Main Isomer): δ =1.28(t, 1H); 3.08(m, 1H); 3.92(m, 2H); 4.23(q, 2H); 6.68(d, 2H); 7.85(t, 1H); 7.90(s, 1H); 8.07(m, 1H); 8.28(m, 1H); 8.52(m, 2H) ppm. | |
| 12 | 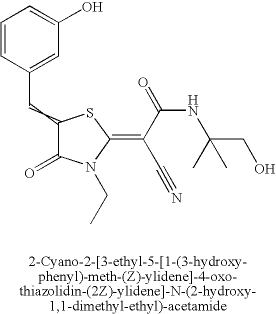 | (DMSO-d6, Stored with K₂CO₃, Main Isomer): δ =1.21(t, 3H); 1.28(s, 6H); 3.35(m, 2H); 4.20(q, 2H); 5.19(t, 1H); 6.86(m, 1H); 6.91(s, 1H); 7.04(m, 1H); 7.08(m, 1H); 7.32(t, 1H); 7.62(s, 1H); 9.93(s, 1H) ppm. | MW: 387.46 MS (ES−) [M − 1]⁻: 386 |
| 13 | 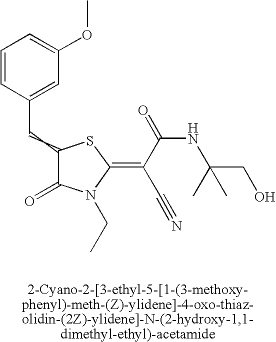 | (DMSO-d6, Stored with K₂CO₃, Main Isomer): δ =1.21-1.30(m, 9H); 3.34(m, 2H); 3.77(s, 3H); 4.21(q, 2H); 5.20(t, 1H); 6.94(s, 1H); 7.08(m, 1H); 7.23(m, 2H); 7.49(t, 1H); 7.71(m, 1H) ppm. | MW: 401.49 MS (ES+) [M + 1]⁺: 402 |
| 14 | 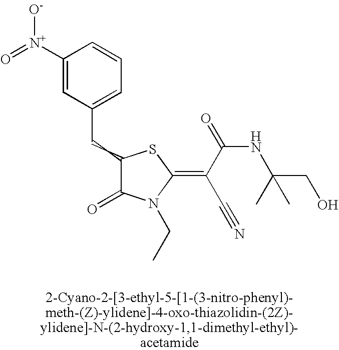 | (DMSO-d6, Stored with K₂CO₃, Main Isomer): δ =1.23-1.30(m, 9H); 3.34(m, 2H); 4.22(q, 2H); 5.21(t, 1H); 6.97(s, 1H); 7.85(t, 1H); 8.07(m, 1H); 8.28(m, 1H); 8.52(m, 1H) ppm. | MW: 416.46 MS (ES−) [M − 1]⁻: 415 |

### EXAMPLES

The following examples describe the biological action of the compounds according to the invention without the action of the compounds being limited to these examples

PLK Enzyme Assay

Recombinant human Plk-1 (6×His) was purified from baculovirus-infected insect cells (Hi5).

10 ng of (produced in a recombinant manner and purified) PLK enzyme is incubated for 90 minutes at room temperature with biotinylated casein and 33P-γ-ATP as a substrate in a volume of 15 μl in 384-well Greiner small-volume microtiter plates (final concentrations in the buffer: 660 ng/ml of PLK; 0.7 μmol of casein, 0.5 μmol of ATP incl. 400 nCi/ml of 33P-γ-ATP; 10 mmol of MgCl2, 1 mmol of MnCI2; 0.01% NP40; 1 mmol of DTT, protease inhibitors; 0.1 mmol of Na2VO3 in 50 mmol of HEPES, pH 7.5). To complete the reaction, 5 μl of stop solution (500 μmol of ATP; 500 mmol of EDTA; 1% Triton X100; 100 mg/ml of streptavidin-coated SPA beads in PBS) is added. After the microtiter plate is sealed by film, the beads are sedimented by centrifuging (10 minutes, 1500 rpm). The incorporation of 33P-γ-ATP in casein is intended as a measurement of enzyme activity by β-counting. The extent of the inhibitor activity is referenced against a solvent control (=uninhibited enzyme activity=0% inhibition) and the mean value of several batches that contained 300 μmol of wortmannin (=completely inhibited enzyme activity=100% inhibition).

Test substances are used in various concentrations (0 μmol, as well as in the range of 0.01-30 μmol). The final concentration of the solvent dimethyl sulfoxide is 1.5% in all batches.

Proliferation Assay

Cultivated human MaTu breast tumour cells were flattened out at a density of 5000 cells/measuring point in a 96-well multititer plate in 200 μl of the corresponding growth medium. After 24 hours, the cells of one plate (zero-point plate) were colored with crystal violet (see below), while the medium of the other plates was replaced by fresh culture medium (200 μl), to which the test substances were added at various concentrations (0 μm, as well as in the range of 0.01-30 μm; the final concentration of the solvent dimethyl sulfoxide was 0.5%). The cells were incubated for 4 days in the presence of test substances. The cell proliferation was determined by coloring the cells with crystal violet: the cells were fixed by adding 20 μl/measuring point of an 11% glutaric aldehyde solution for 15 minutes at room temperature. After three washing cycles of the fixed cells with water, the plates were dried at room temperature. The cells were colored by adding 100 μl/measuring point of a 0.1% crystal violet solution (pH was set at 3 by adding acetic acid). After three washing cycles of the colored cells with water, the plates were dried at room temperature. The dye was dissolved by adding 100 μl/measuring point of a 10% acetic acid solution. The extinction was determined by photometry at a wavelength of 595 nm. The change of cell growth, in percent, was calculated by standardization of the measured values to the extinction values of the zero-point plate (=0%) and the extinction of the untreated (0 μm) cells (=100%).

TABLE 1
Assay Data:

| | | | Inhibition of |
|---|---|---|---|
| | | PLK-1 | the Tumour |
| | | IC50 | Cell |
| Ex- | | [nM] | Proliferation |
| ample | | Inhi- | (MaTu) |
| No. | Structure | bition | IC50 [μM] |
| 10 | 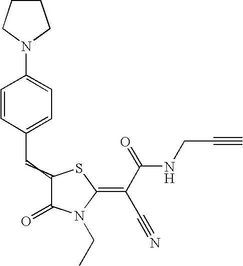 | 23 | Not Determined |
| 3 | 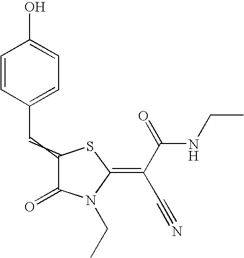 | 230 | Not Determined |
| 8 | 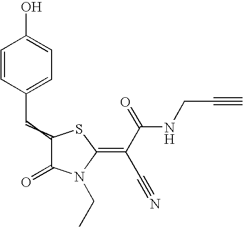 | 39 | 3.2 |

It thus can be seen from Table 1 that the compounds of general formula (I) exhibit nanomolar inhibition both on the enzyme and in the proliferation test.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

The entire disclosures of all applications, patents and publications, cited herein and of corresponding German application No. 102 005 020 105.9, filed Apr. 25, 2005, and U.S. Provisional Application Ser. No. 60/676,947, filed May 3, 2005, are incorporated by reference herein.

The preceding examples can be repeated with similar success by substituting the generically or specifically described reactants and/or operating conditions of this invention for those used in the preceding examples.

1. A compound of general formula I: 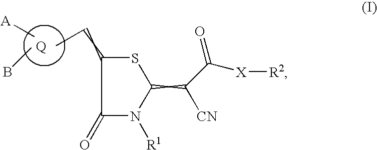
in which:
Q stands for aryl,
A and B, independently of one another, stand for hydrogen, halogen, hydroxy, —NR³R⁴, cyano or nitro,
or
for C₁-C₄-alkyl, C₁-C₆-alkoxy or C₃-C₆-heterocycloalkyl that optionally is substituted in one or more places, in the same way or differently, with halogen, hydroxy, C₃-C₆-heterocycloalkyl or with the group —NR³R⁴ or —CO(NR³)-M, whereby the heterocycloalkyl itself optionally can be interrupted by one or more nitrogen, oxygen and/or sulfur atoms and/or optionally can be interrupted by one or more —C(O)— or —SO₂— groups in the ring and/or optionally one or more double bonds can be contained in the ring and/or the ring itself optionally can be substituted in one or more places, in the same way or differently, with C₁-C₆-alkyl, C₃-C₆-cycloalkyl, C₁-C₆-hydroxyalkyl or with the group —NR³R⁴,
or
for —NR³C(O)-L, —NR³C(O)-NR³-L, —COR⁶, —O—(CH₂)ₚR⁶, —CO(NR³)-M, —NR³(CS)NR³R⁴, —NR³SO₂-M, —SO₂—NR³R⁴, —SO₂(NR³)-M or —O—(CH₂)ₚaryl,
P stands for an integer of 0, 1, 2, 3, or 4,
L stands for C₁-C₆-alkyl or C₃-C₆-heterocycloalkyl that optionally is substituted in one or more places, in the same way or differently, with C₁-C₆-hydroxyalkoxy, C₁-C₆-alkoxyalkoxy, C₃-C₆-heterocycloalkyl or with the group —NR³R⁴, whereby the heterocycloalkyl itself optionally can be interrupted by one or more nitrogen, oxygen and/or sulfur atoms and/or optionally can be interrupted by one or more —C(O)— or —SO₂— groups in the ring and/or optionally one or more double bonds can be contained in the ring, and/or the ring itself optionally can be substituted in one or more places, in the same way or differently, with C₁-C₆-alkyl, C₃-C₆-cycloalkyl, C₁-C₆-hydroxyalkyl or with the group —NR³R⁴,
M stands for C₁-C₆-alkyl that optionally is substituted in one or more places, in the same way or differently, with the group —NR³R⁴ or C₃-C₆-heterocycloalkyl, x stands for —NH— or —NR⁵—,
R¹ stands for C₁-C₄-alkyl, C₃-cycloalkyl, allyl or propargyl that optionally is substituted in one or more places, in the same way or differently, with halogen,
R² stands for hydrogen or for C₁-C₆-alkyl, C₁-C₆-alkoxy, C₁-C₆-alkenyl, C₁-C₆-alkynyl, C₃-C₆-cycloalkyl, C₃-C₆-heterocycloalkyl, aryl or heteroaryl that optionally is substituted in one or more places, in the same way or differently, with halogen, hydroxy, cyano, C₁-C₆-alkyl, C₁-C₆-alkoxy, C₁-C₆-hydroxyalkyl, C₃-C₆-cycloalkyl, C₃-C₆-heterocycloalkyl, C₁-C₆-alkynyl, aryl, aryloxy, heteroaryl or with the group-S—C₁-C₆-alkyl, —COR⁶, —NR³R⁴, —NR³C(O)-L or —NR³COOR⁷,
whereby the heterocycloalkyl itself optionally can be interrupted by one or more nitrogen, oxygen and/or sulfur atoms and/or optionally can be interrupted by one or more —C(O)— or —SO₂ groups in the ring and/or optionally one or more double bonds can be contained in the ring,
and whereby aryl, heteroaryl, C₃-C₆-cycloalkyl- and/or the C₃-C₆-heterocycloalkyl ring in each case itself optionally can be substituted in one or more places, in the same way or differently, with cyano, halogen, hydroxy, C₁-C₆-alkyl, C₁-C₆-hydroxyalkyl, or C₁-C₆-alkoxy, C₃-C₆-cycloalkyl, C₃-C₆-heterocycloalkyl, aryl, benzyl or heteroaryl that optionally is substituted in one or more places, in the same way or differently, with halogen,
or
for the group —NR³R⁴, —NR³C(O)-L, or —NR³(CS)NR³R⁴,
or
R² and R⁵ together form a C₃-C₆-heterocycloalkyl ring, which is interrupted at least once by nitrogen and optionally can be interrupted in one or more places by oxygen or sulfur and/or optionally can be interrupted by one or more —C(O)— or —SO₂— groups in the ring and/or optionally one or more double bonds can be contained in the ring,
and/or the ring itself optionally can be substituted in one or more places, in the same way or differently, with cyano, halogen, hydroxy, C₁-C₆-alkyl, C₃-C₆-cycloalkyl, C₁-C₆-hydroxyalkyl, C₁-C₆-alkoxyalkyl or with the group —NR³R⁴ or —COR⁶,
and/or can be substituted with aryl or heteroaryl that optionally is substituted in one or more places, in the same way or differently, with halogen, C₁-C₆-alkoxy or with the group —COR⁶,
R³ and R⁴, independently of one another, stand for hydrogen or for C₁-C₆-alkyl, C₁-C₆-alkoxy, —CO—C₁-C₆-alkyl or aryl that optionally is substituted in one or more places, in the same way or differently, with halogen, hydroxy, C₃-C₆-heterocycloalkyl, C₁-C₆-hydroxyalkoxy or with the group —NR³R⁴, whereby the heterocycloalkyl itself optionally can be interrupted by one or more nitrogen, oxygen and/or sulfur atoms and/or optionally can be interrupted by one or more —C(O)— or —SO₂— groups in the ring and/or optionally one or more double bonds can be contained in the ring, and
whereby the C₃-C₆-heterocycloalkyl ring itself optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group —NR³R⁴ or —CO—NR³R⁴,
or
R³ and R⁴ together form a C₃-C₆-heterocycloalkyl ring, which is interrupted at least once by nitrogen and optionally can be interrupted in one or more places by oxygen or sulfur and/or optionally can be interrupted by one or more —C(O)— or —SO₂— groups in the ring and/or optionally one or more double bonds can be contained in the ring, and/or the heterocycloalkyl ring itself optionally can be substituted in one or more places, in the same way or differently, with C₁-C₆-alkyl, C₃-C₆-cycloalkyl, C₁-C₆-hydroxyalkyl, C₁-C₆-alkoxyalkyl, cyano, hydroxy or with the group —NR³R⁴,
R⁵ stands for C₁-C₆-alkyl, C₁-C₆-alkenyl, or C₁-C₆-alkynyl that optionally is substituted in one or more places, in the same way or differently, with halogen, hydroxy, cyano, C₁-C₆-alkoxy, C₃-C₆-cycloalkyl, C₃-C₆-heterocycloalkyl, or with the group —NR³R⁴,
whereby the heterocycloalkyl itself optionally can be interrupted by one or more nitrogen, oxygen and/or sulfur atoms and/or optionally can be interrupted by one or more —C(O)— or —SO₂ groups in the ring and/or optionally one or more double bonds can be contained in the ring, and
whereby the C₃-C₆-heterocycloalkyl ring itself in each case optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group —NR³R⁴ or —CO—NR³R⁴,
R⁶ stands for hydroxy, C₁-C₆-alkyl, C₁-C₆-alkoxy or the group —NR³R⁴,
R⁷ stands for —CH₂)ₙ-aryl or —CH₂)n-heteroaryl, and
n stands for an integer of 1, 2, 3, 4, 5 or 6,
as well as the solvates, hydrates, stereoisomers, diastereomers, enantiomers and salts thereof.

2. The compound of general formula I according to claim 1, in which:
Q stands for phenyl,
A and B, independently of one another, stand for hydrogen, halogen, hydroxy, —NR³R⁴, cyano or nitro,
or
for C₁-C₄-alkyl, C₁-C₆-alkoxy or C₃-C₆-heterocycloalkyl that optionally is substituted in one or more places, in the same way or differently, with halogen, hydroxy, C₃-C₆-heterocycloalkyl or with the group —NR³R⁴ or —CO(NR³)-M,
whereby the heterocycloalkyl itself optionally can be interrupted by one or more nitrogen, oxygen and/or sulfur atoms, and/or optionally can be interrupted by one or more —C(O)— or —SO₂ groups in the ring and/or optionally one or more double bonds can be contained in the ring and/or the ring itself optionally can be substituted in one or more places, in the same way or differently, with C₁-C₆-alkyl, C₃-C₆-cycloalkyl, C₁-C₆-hydroxyalkyl or with the group —NR³R⁴,
or
for —NR³C(O)-L, —NR³C(O)-NR³-L, —COR⁶, —O—(CH₂)ₚR⁶, —CO(NR³)-M,
—NR³(CS)NR³R⁴, —NR³SO₂-M, —SO₂—NR³R⁴, —SO₂(NR³)-M or —O—(CH₂)ₚaryl,
p stands for an integer of 0, 1, 2, 3, or 4,
L stands for C₁-C₆-alkyl or C₃-C₆-heterocycloalkyl that optionally is substituted in one or more places, in the same way or differently, with C₁-C₆-hydroxyalkoxy, C₁-C₆-alkoxyalkoxy, C₃-C₆-heterocycloalkyl or with the group —NR³R⁴,
whereby the heterocycloalkyl itself optionally can be interrupted by one or more nitrogen, oxygen and/or sulfur atoms and/or optionally can be interrupted by one or more —C(O)— or —SO₂ groups in the ring and/or optionally one or more double bonds can be contained in the ring and/or the ring itself optionally can be substituted in one or more places, in the same way or differently, with C₁-C₆-alkyl, C₃-C₆-cycloalkyl, C₁-C₆-hydroxyalkyl or with the group —NR³R⁴,
M stands for C₁-C₆-alkyl that optionally is substituted in one or more places, in the same way or differently, with the group —NR³R⁴ or C₃-C₆-heterocycloalkyl,
X stands for —NH— or —NR⁵—,
R¹ stands for C₁-C₄-alkyl, C₃-cycloalkyl, allyl or propargyl that optionally is substituted in one or more places, in the same way or differently, with halogen,
R² stands for hydrogen or for C₁-C₆-alkyl, C₁-C₆-alkoxy, C₁-C₆-alkenyl, C₁-C₆-alkynyl, C₃-C₆-cycloalkyl, C₃-C₆-heterocycloalkyl, aryl or heteroaryl that optionally is substituted in one or more places, in the same way or differently, with halogen, hydroxy, cyano, C₁-C₆-alkyl, C₁-C₆-alkoxy, C₁-C₆-hydroxyalkyl, C₃-C₆-cycloalkyl, C₃-C₆-heterocycloalkyl, C₁-C₆-alkynyl, aryl, aryloxy, heteroaryl or with the group —S—C₁-C₆-alkyl, —COR₆, —NR³R⁴, —NR³C(O)-L or —NR³COOR⁷,
whereby the heterocycloalkyl itself optionally can be interrupted by one or more nitrogen, oxygen and/or sulfur atoms and/or optionally can be interrupted by one or more —C(O)— or —SO₂ groups in the ring and/or optionally one or more double bonds can be contained in the ring,
and whereby aryl, heteroaryl, C₃-C₆-cycloalkyl and/or the C₃-C₆-heterocycloalkyl ring in each case itself optionally can be substituted in one or more places, in the same way or differently, with cyano, halogen, hydroxy, C₁-C₆-alkyl, C₁-C₆-hydroxyalkyl; C₁-C₆-alkoxy, C₃-C₆-cycloalkyl, C₃-C₆-heterocycloalkyl, aryl, benzyl or heteroaryl that optionally is substituted in one or more places, in the same way or differently, with halogen,
or
for the group —NR³R⁴, —NR³C(O)-L, —NR³(CS)NR³R⁴,
or
R² and R⁵ together form a C₃-C₆-heterocycloalkyl ring, which is interrupted at least once by nitrogen and optionally can be interrupted in one or more places by oxygen or sulfur and/or optionally can be interrupted by one or more —C(O)— or —SO₂— groups in the ring and/or optionally one or more double bonds can be contained in the ring,
and/or the ring itself optionally can be substituted in one or more places, in the same way or differently, with cyano, halogen, hydroxy, C₁-C₆-alkyl, C₃-C₆-cycloalkyl, C₁-C₆-hydroxyalkyl, C₁-C₆-alkoxyalkyl or with the group —NR³R⁴ or —COR⁶ and/or can be substituted with aryl or heteroaryl that optionally is substituted in one or more places, in the same way or differently, with halogen, C₁-C₆-alkoxy or with the group —COR⁶,
R³ and R⁴, independently of one another, stand for hydrogen or for C₁-C₆-alkyl, C₁-C₆-alkoxy, —CO—C₁-C₆-alkyl or aryl that optionally is substituted in one or more places, in the same way or differently, with halogen, hydroxy, C₃-C₆-heterocycloalkyl, C₁-C₆-hydroxyalkoxy or with the group —NR³R⁴, whereby the heterocycloalkyl itself optionally can be interrupted by one or more nitrogen, oxygen and/or sulfur atoms and/or optionally can be interrupted by one or more —C(O)— or —SO₂— groups in the ring and/or optionally one or more double bonds can be contained in the ring, and
whereby the C₃-C₆-heterocycloalkyl ring itself in each case optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl or with the group —NR³R⁴ or —CO—NR³R⁴,
or
R³ and R⁴ together form a C₃-C₆-heterocycloalkyl ring, which is interrupted at least once by nitrogen and optionally can be interrupted in one or more places by oxygen or sulfur and/or optionally can be interrupted by one or more —C(O)— or —SO₂— groups in the ring and/or optionally one or more double bonds can be contained in the ring
and/or the heterocycloalkyl ring itself optionally can be substituted in one or more places, in the same way or differently, with C₁-C₆-alkyl, C₃-C₆-cycloalkyl, C₁-C₆-hydroxyalkyl, C₁-C₆-alkoxyalkyl, cyano, hydroxy or with the group —NR³R⁴,
R⁵ stands for C₁-C₆-alkyl, C₁-C₆-alkenyl or C₁-C₆-alkynyl that optionally is substituted in one or more places, in the same way or differently, with halogen, hydroxy, cyano, C₁-C₆-alkoxy, C₃-C₆-cycloalkyl, C₃-C₆-heterocycloalkyl or with the group —NR³R⁴, whereby the heterocycloalkyl itself optionally can be interrupted by one or more nitrogen, oxygen and/or sulfur atoms and/or optionally can be interrupted by one or more —C(O)— or —SO₂— groups in the ring and/or optionally one or more double bonds can be contained in the ring and
whereby the C₃-C₆-heterocycloalkyl ring itself in each case optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group —NR³R⁴ or —CO—NR³R⁴,
R⁶ stands for hydroxy, C₁-C₆-alkyl, C₁-C₆-alkoxy or the group —NR³R⁴,
R⁷ stands for —(CH₂)ₙ-aryl or —(CH₂)ₙ-heteroaryl and
n stands for an integer of 1, 2, 3, 4, 5 or 6,
as well as the stereoisomers, diastereomers, enantiomers and salts thereof.

3. The compound of general formula I according to claim 1, in which:
Q stands for phenyl,
A and B, independently of one another, stand for hydrogen, halogen, or for C₁-C₄-alkyl or pyrrolidinyl that optionally is substituted in one or more places, in the same way or differently, with halogen, hydroxy or with the group —NR³R⁴ or —CO(NR³)-M,
M stands for C₁-C₆-alkyl that optionally is substituted in one or more places, in the same way or differently, with the group —NR³R⁴ or C₃-C₆-heterocycloalkyl,
X stands for —NH—,
R¹ stands for C₁-C₄-alkyl that optionally is substituted in one or more places, in the same way or differently, with halogen,
R² stands for hydrogen or for C₁-C₆-alkyl or C₁-C₆-alkynyl that optionally is substituted in one or more places, in the same way or differently, with halogen, cyano, or C₁-C₆-alkoxy,
R³ and R⁴, independently of one another, stand for hydrogen or for C₁-C₆-alkyl, C₁-C₆-alkoxy, —CO—C₁-C₆-alkyl or aryl that optionally is substituted in one or more places, in the same way or differently, with halogen, hydroxy, C₃-C₆-heterocycloalkyl, C₁-C₆-hydroxyalkoxy or with the group —NR³R⁴, whereby the heterocycloalkyl itself optionally can be interrupted by one or more nitrogen, oxygen and/or sulfur atoms, and/or optionally can be interrupted by one or more —C(O)— or —SO₂— groups in the ring and/or optionally one or more double bonds can be contained in the ring, and
whereby the C₃-C₆-heterocycloalkyl ring itself optionally can be substituted in one or more places, in the same way or differently, with cyano, halogen, C₁-C₆-alkyl, C₁-C₆-hydroxyalkyl, C₁-C₆-alkoxy, C₃-C₆-cycloalkyl, or with the group —NR³R⁴ or —NR³R⁴,
or
R³ and R⁴ together form a C₃-C₆-heterocycloalkyl ring, which is interrupted at least once by nitrogen and optionally is interrupted in one or more places by oxygen or sulfur and/or optionally can be interrupted by one or more —C(O)— or —SO₂— groups in the ring and/or optionally one or more double bonds can be contained in the ring, and/or the heterocycloalkyl ring itself optionally can be substituted in one or more places, in the same way or differently, with C₁-C₆-alkyl, C₃-C₆-cycloalkyl, C₁-C₆-hydroxyalkyl, C₁-C₆-alkoxyalkyl, cyano, hydroxy or with the group —NR³R⁴, and
R⁶ stands for hydroxy or C₁-C₆-alkoxy,
as well as the solvates, hydrates, stereoisomers, diastereomers, enantiomers and salts thereof.

4. The compound of general formula I according to claim 1, in which:
Q stands for phenyl,
A and B, independently of one another, stand for hydrogen, halogen, hydroxy, methoxy or pyrrolidinyl,
X stands for —NH—,
R¹ stands for ethyl,
R² stands for ethyl or propynyl,
as well as the solvates, hydrates, stereoisomers, diastereomers, enantiomers and salts thereof

5. The compound of general formula I according to claim 1, which is selected from the group consisting of:
2-Cyano-N-ethyl-2-[3-ethyl-4-oxo-5-[1-(4-pyrrolidin-1-yl-phenyl)-meth-(Z)-ylidene]-thiazolidin-(2Z)-ylidene]-acetamide; 2-Cyano-N-ethyl-2-[3-ethyl-5-[1-(3-hydroxy-phenyl)-meth-(Z)-ylidene]-4-oxo-thiazolidin-(2Z)-ylidene]-acetamide; 2-Cyano-N-ethyl-2-[3-ethyl-5-[1-(4-hydroxy-phenyl)-meth-(Z)-ylidene]-4-oxo-thiazolidin-(2Z)-ylidene]-acetamide; 2-[5-[1-(4-Bromo-phenyl)-meth-(Z)-ylidene]-3-ethyl-4-oxo-thiazolidin-(2Z)-ylidene]-2-cyano-N-ethyl-acetamide; 2-Cyano-N-ethyl-2-[3-ethyl-5-[1-(4-hydroxy-3-methoxy-phenyl)-meth-(Z)-ylidene]-4-oxo-thiazolidin-(2Z)-ylidene]-acetamide; 2-Cyano-N-ethyl-2-[3-ethyl-5-[1-(3-methoxy-phenyl)-meth-(Z)-ylidene]-4-oxo-thiazolidin-(2Z)-ylidene]-acetamide; 2-Cyano-2-[3-ethyl-5-[1-(3-hydroxy-phenyl)-meth-(Z)-ylidene]-4-oxo-thiazolidin-(2Z)-ylidene]-N-prop-2-ynyl-acetamide; 2-Cyano-2-[3-ethyl-5-[1-(4-hydroxy-phenyl)-meth-(Z)-ylidene]-4-oxo-thiazolidin-(2Z)-ylidene]-N-prop-2-ynyl-acetamide; 2-Cyano-2-[3-ethyl-5-[1-(4-hydroxy-3-methoxy-phenyl)-meth-(Z)-ylidene]-4-oxo-thiazolidin-(2Z)-ylidene]-N-prop-2-ynyl-acetamide; 2-Cyano-2-[3-ethyl-4-oxo-5-[1-(4-pyrrolidin-1-yl-phenyl)-meth-(Z)-ylidene]-thiazolidin-(2Z)-ylidene]-N-prop-2-ynyl-acetamide; 2-Cyano-2-[3-ethyl-5-[1-(3-nitro-phenyl)-meth-(Z)-ylidene]-4-oxo-thiazolidin-(2Z)-ylidene]-N-prop-2-ynyl-acetamide; 2-Cyano-2-[3-ethyl-5-[1-(3-hydroxy-phenyl)-meth-(Z)-ylidene]-4-oxo-thiazolidin-(2Z)-ylidene]-N-(2-hydroxy-1,1-dimethyl-ethyl)-acetamide; 2-Cyano-2-[3-ethyl-5-[1-(3-methoxy-phenyl)-meth-(Z)-ylidene]-4-oxo-thiaz-olidin-(2Z)-ylidene]-N-(2-hydroxy-1,1-dimethyl-ethyl)-acetamide; and 2-Cyano-2-[3-ethyl-5-[1-(3-nitro-phenyl)-meth-(Z)-ylidene]-4-oxo-thiazolidin-(2Z)-ylidene]-N-(2-hydroxy-1,1-dimethyl-ethyl)-acetamide.

6. A method of preparing a compound of general formula (I) according to claim 1, wherein a carboxylic acid of general formula (3): 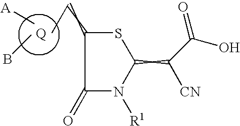
(3)
is allowed to react with a compound of general formula (3′):
R²—XH (3′);
thus providing a compound of general formula (I): 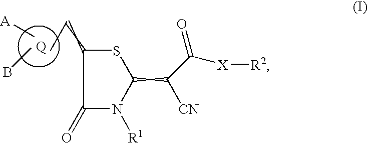
in which formulae (3), (3′) and (I), the definitions of substituents R¹, R², A, B, Q, and X being according to claim 1.

7. The method according to claim 6, wherein said carboxylic acid of general formula (3) is prepared by allowing an allyl ester of general formula (2): 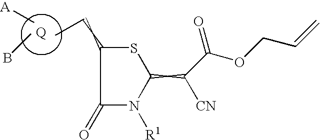
(2)
to be saponified in the presence of Pd-tetrakis-triphenylphosphine and barbituric acid, in which formula (2) the definitions of substituents R¹, A, B, and Q being according to above.

8. The method according to claim 7, wherein said allyl ester of general formula (2) is prepared by allowing a thiazolidinone of general formula (1): 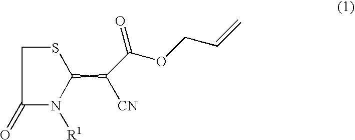
to be condensed with an aldehyde of general formula (1′): 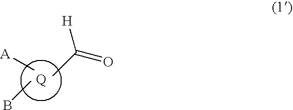
in which formulae (1) and (1′), the definitions of substituents R¹, A, B, and Q being according to above.

9. A method of preparing a compound of general formula (I) according to claim 1, wherein a thiazolidinone of general formula (5): 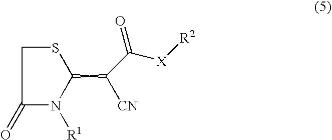
is allowed to be condensed with an aldehyde of general formula (1′): 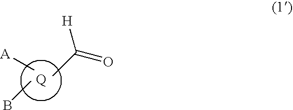
thus providing a compound of general formula (I): 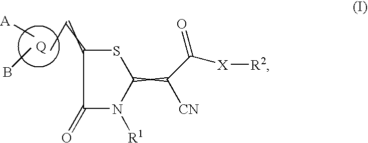
in which formulae (5), (1′) and (I), the definitions of substituents R¹, R², A, B, Q, and X being according to claim 1.

10. The method according to claim 9, wherein said thiazolidinone of general formula (5) is prepared by allowing a carboxylic acid of general formula (4): 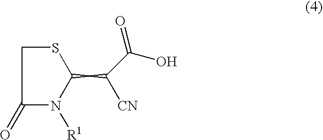
to react with a compound of general formula (3′):
R²—XH (3′);
in which formulae (4) and (3′), the definitions of substituents R¹, R² and X being according to above.

11. The method according to claim 10, wherein said carboxylic acid of general formula (4) is prepared by allowing a thiazolidinone of general formula (1): 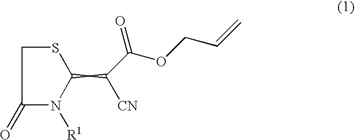
to be saponified in the presence of Pd-tetrakis-triphenylphosphine and barbituric acid, in which formula (1) the definition of the substituent R¹ being according to above.

12. Use of a compound of general formula I, or of a compound as obtained according to claim 6, for the production of a pharmaceutical agent for treating cancer, auto-immune diseases, chemotherapy agent-induced alopecia and mucositis, cardiovascular diseases, infectious diseases, nephrological diseases, chronic and acute neurodegenerative diseases and viral infections.

13. Use according to claim 12, wherein cancer is defined as solid tumours and leukemia; auto-immune diseases are defined as psoriasis, alopecia and multiple sclerosis; cardiovascular diseases are defined as stenoses, arterioscleroses, and restenoses; infectious diseases are defined as diseases that are caused by unicellular parasites; nephrological diseases are defined as glomerulonephritis; chronic neurodegenerative diseases are defined as Huntington's disease, amyotrophic lateral sclerosis, Parkinson's disease, AIDS dementia and Alzheimer's disease; acute neurodegenerative diseases are defined as ischemias of the brain and neurotraumas; and viral infections are defined as cytomegalic infections, herpes, hepatitis B and C, and HIV diseases.

14. A pharmaceutical agent, characterized in that it contains at least one compound according to Formula I, or as obtained by a method according to claim 6.

15. The pharmaceutical agent according to claim 14 for treating cancer, autoimmune diseases, cardiovascular diseases, infectious diseases, nephrological diseases, neurodegenerative diseases and viral infections.

16. A compound according to Formula I, or as obtained according to claim 6, or a pharmaceutical agent containing it, together with a suitable formulation substance and/or vehicle.

17. Use of a compound of general formula I according to Formula I, or as obtained by a method according to claim 6, or of a pharmaceutical agent containing it, as an inhibitor of a polo-like kinase.

18. Use according to claim 17, wherein said kinase is Plk1, Plk2, Plk3 or Plk4.

19. Use of a compound of general formula I according to Formula I, or as obtained according to claim 6, in the form of a pharmaceutical preparation for enteral, parenteral and oral administration.
